SEQUENCE LISTING (4 sequences)

SEQ ID NO: 1 - DNA, 59 bp, Artificial Sequence
  source: /note=“Description of Artificial Sequence Synthetic oligonucleotide”
tgcgtagcgatgactagctgctgatcgatattagctagcatcagcgatcgatacgagca

SEQ ID NO: 2 - DNA, 59 bp, Artificial Sequence
  source: /note=“Description of Artificial Sequence Synthetic oligonucleotide”
tgctcgtatcgatcgctgatgctagctaatatcgatcagcagctagtcatcgctacgca

SEQ ID NO: 3 - DNA, 23 bp, Artificial Sequence
  source: /note=“Description of Artificial Sequence Synthetic primer”
tgcgtagcgatgactagctgctg

SEQ ID NO: 4 - DNA, 23 bp, Artificial Sequence
  source: /note=“Description of Artificial Sequence Synthetic primer”
tgctcgtatcgatcgctgatgct

(19) United States
(12) Patent Application Publication
(10) Pub. No.: US 2015/0044666 A1
(43) Pub. Date: Feb. 12, 2015

(54) NON-EQUILIBRIUM TWO-SITE ASSAYS FOR LINEAR, ULTRASENSITIVE ANALYTE DETECTION

(71) Applicant: IRIS International Inc., Chatsworth, CA (US)

(72) Inventors: Edward Jablonski, Escondido, CA (US); Thomas H. Adams, Rancho Santa Fe, CA (US); David Driver, Solana Beach, CA (US); Thomas Brendan Ryder, Encinitas, CA (US)

(21) Appl. No.: 14/371,691

(22) PCT Filed: Jan. 11, 2013
(86) PCT No.: PCT/US2013/021320

§ 371 Date: Jul. 10, 2014

Related U.S. Application Data
(60) Provisional application No. 61/586,669, filed on Jan. 13, 2012.

(51) Int. Cl. C12Q 1/68

(52) U.S. Cl. CPC C12Q 1/6804
USPC 435/5; 435/6.12

(57) ABSTRACT

Methods and kits related to non-equilibrium, ultrasensitive two-site assays for detecting analytes are provided. In one aspect, a two-site assays for detecting analytes under non-equilibrium analyte binding conditions, using low concentrations of reporter specificity molecule (e.g., reporter antibody) and kits for performing the same is provided. In another aspect, methods for selecting antibodies or specificity molecules with low dissociation constants for use as reporter antibodies in non-equilibrium two-site immunoassays, including two-site immuno-PCR assays, and assays performed with those antibodies, are also provided.

### RELATED APPLICATION

The present application is a U.S. National Phase application of PCT/US2013/021320, filed on Jan. 11, 2013 which claims priority of U.S. provisional application Ser. No. 61/586,669, filed on Jan. 13, 2012, the contents of which are incorporated by reference herein in their entirety.

This application includes a Sequence Listing as a text file named “SequenceListing_{—}87904-913405.txt” created Jul. 9, 2014, and containing 4,096 bytes. The material contained in this text file is incorporated by reference in its entirety for all purposes.

### FIELD OF THE INVENTION

This invention relates to non-equilibrium, ultrasensitive two-site assays for detecting analytes. In one aspect this invention relates to two-site assays for detecting analytes under non-equilibrium analyte binding conditions, using low concentrations of reporter specificity molecule, e.g., reporter antibody, and kits for performing the same. This invention also relates in another aspect to methods for selecting antibodies or specificity molecules with low dissociation constants for use as reporter antibodies in non-equilibrium two-site immunoassays, including two-site immuno-PCR assays, and assays performed with those antibodies.

### BACKGROUND

Immunoassays have gained wide use in medical diagnosis since Berson & Yalow first described an immunoassay for determining the concentration of antigens through binding of the antigens to specific antibodies in 1959. Since Berson & Yalow's description of a competitive radioimmune assay for plasma insulin, immunoassays using non-isotopic labels have also been reported.

As an example, enzyme-linked immunosorbent assays (ELISA) using enzyme catalyzed signal production have been reported. In ELISA assays, target analytes were immobilized by non-specific adsorption to a solid surface. After washing to remove excess antigen and blocking the remaining sites on the solid surface, the immobilized antigen was contacted with a primary antibody to form an immune complex on the solid surface. Excess antibody was removed by washing. The primary antibody was then detected via a reaction catalyzed by an enzyme directly conjugated to the primary antibody (direct ELISA), or to a second anti-antibody, which was subsequently contacted with the solid surface (indirect ELISA). The enzyme activity associated with the solid surface was proportional to the amount of bound antigen present and was measured, for example, by using a chromogenic substrate for the enzyme. ELISA assays can detect target analytes present in amounts of a few million molecules per milliliter of sample. However, almost all protein analytes are present in plasma below the threshold of detection for an ELISA assay. Only 1% of proteins are currently available in levels high enough to be useful diagnostically. See Science 302:21 (2003), pp 1316-1318.

The development of monoclonal antibodies by Köhler and Milstein led to the use of single specificity antibodies in diagnostic applications. For example, sandwich immunoassays using monoclonal antibodies with equilibrium binding constants of less than 10⁻⁸ were described by David and Greene in U.S. Pat. No. 4,486,530. Two-site immunoassay methods using a single incubation of the analyte, the labeled reporter antibody, and the capture antibody bound to a solid support have also been described. See Blakemore, U.S. Pat. No. 4,244,940

Sandwich assays run in either a reverse or forward orientation have been reported. In a reverse sandwich immunoassay, the antigen was first contacted with the capture antibody on the solid support, to form a capture antibody-antigen complex followed by washing, and then a labeled reporter antibody was then contacted with the complex. The resulting sandwich immune complex was bound to the surface of the solid support and consisted of the antigen combined with the two antibodies. Excess antigen and antibodies were removed by repeatedly washing the support. Following the washes, the bound reporter antibody was measured via the detection molecule as an indication of the amount of antigen present.

In the forward sandwich orientation, the reporter antibody-label conjugate was contacted with a sample containing an analyte to form a first immune complex. The immune complex was then captured onto a solid support coated with the second capture antibody, forming an immobilized two-site immune complex. The solid support to which the immobilized two-site immune complex was bound was washed several times to remove excess reporter antibody-label conjugate, and bound reporter antibody was measured.

Immuno-PCR, first described by Sano and Cantor in 1992 (Sano et al., Science, 1992, 258:120-22), combined the selectivity of detection of an antigen by immunoassay with some of the detection sensitivity of PCR. In immuno-PCR, a strand of DNA used as the label was detected by Polymerase Chain Reaction (PCR) amplification (Mullis K B, 1987). Amplification of the DNA label permitted much higher sensitivity than that obtained using an enzyme-linked signal generation system.

However, because PCR can detect single molecules of target DNA, the sequential addition of each immunoassay component in an immuno-PCR assay required extensive washing to reduce non-specifically bound material. Although Sano et al. achieved high sensitivity, they used a pure system and a cumbersome, multi-step format with intermediate washing steps, which was impractical for high throughput medical applications.

Immuno-PCR can be performed in a sandwich immunoassay format similar to that of an indirect ELISA assay. Examples of sandwich immuno-PCR assays have been reported in the literature (see, e.g., Joerger et al., Clin Chem, 1995, 41:1371-77; Hendrickson et al., Nucleic Acids Res, 1995, 23:522-529). Detection limits for the sandwich Immuno-PCR assay format exceeded the detection limits of ELISA assays, due to exponential amplification of the signal DNA. Stringent washing, however, was required to reduce the number of non-specifically bound DNA-labeled reporter antibodies.

The sandwich format of Immuno-PCR has also been used to demonstrate the detection of multiple antigens (“multiplexing”) (see e.g., U.S. Pat. No. 5,985,548). DNA labels of different length were each coupled to reporter antibodies recognizing distinct antigens using a 5′ N-hydroxysuccinimide ester (NHS). The reporter antibody-DNA conjugates bound to a solid support coated with the various antigens. The multiple DNA labels were detected simultaneously in the assay based on the sizes of the respective PCR amplification products, which had been designed to allow resolution from each other by gel electrophoresis.

### SUMMARY OF THE INVENTION

In one aspect this invention relates to a two-site assay which can detect analyte present in a sample at levels or concentrations of less than about 10 pg/mL, or other lower threshold amounts of analyte. The specificity molecules used in the two-site assays may be antibodies, receptors, ligands, or other molecules capable of specifically binding to the analyte. In some embodiments the assays are run in the forward orientation. In another aspect, the two-site assay is a forward immunoassay which employs two antibodies which specifically recognize the antigen to be detected. A “capture” specificity molecule or antibody is used to coat the surface of a solid support, such as a microtiter well, bead or particle. A “reporter” specificity molecule or antibody is labeled with a high sensitivity detection molecule, such as a nucleic acid molecule, a chemiluminescent label, nanoparticle, or other suitable label. In certain aspects, the two-site immunoassay uses a DNA molecule as the label.

In some aspects of this invention, the assay is a forward immuno-PCR assay which comprises contacting a reporter MAb-DNA conjugate with a sample containing an analyte to form a first immune complex. The immune complex is then captured onto a solid support coated with a second MAb (capture MAb), forming an immobilized two-site immune complex. The solid support to which the immobilized two-site immune complex is bound is washed to remove excess reporter MAb-DNA conjugate. In one embodiment, the solid support is washed several times. Following the washing of the bound two-site immune complex, the bound reporter antibody is measured via the detection label as an indication of the amount of antigen present.

In some aspects the methods of this invention as described herein relate to ultrasensitive, forward, two-site immunoassays, which utilize highly sensitive labels such as nucleic acid labels for detection, and which are capable of detecting analytes present in a sample at concentrations ranging from at least as low as about 0.01 pg/mL to about 10, 15 or 30 pg/mL. In related aspects, the methods of this invention can detect analytes present in samples at concentrations as low as about 100 fM, 10 fM, or even as low as 0.1 fM. The assays of this invention may also exhibit a wide dynamic range over at least two or three orders of magnitude, exhibiting linearity from the limit of quantitation up to, for example, over about 1000 pg/mL or 2000 pg/mL, or over about 7.5 or 15 pM.

The assays may employ a soluble reporter monoclonal antibody (MAb) which specifically binds to the analyte, where the reporter antibody is labeled with a high sensitivity label, to form a MAb-label conjugate. The label may be, for example, an assay-specific nucleic acid sequence. In one aspect, the nucleic acid sequence is a double-stranded DNA sequence, but other detectable nucleic acids or modified nucleic acids may be used as labels in the assays of this invention. In other aspects, the label may be, for example, a chemiluminescent label, a nanoparticle label, or other label which generates a signal sufficient to detect picomolar or femtomolar concentrations of analyte. The assays of this invention are also useful in a multiplex format in which the reporter antibody for each analyte is labeled with a different DNA marker, and can each be detected in a multiplex PCR format.

The second capture MAb is specific for a second site on the analyte. In one embodiment the capture MAb is coated onto a solid surface. In some embodiments, the solid surface may be glass particles or fibers, nanoparticles, paramagnetic microparticles, or other suitable solid, microparticle, or microbubble support. In some aspects, the capture MAb-solid surface may be suitable for use with instrumentation capable of performing high throughput, automated solid phase immunoassays. In a preferred embodiment, the label does not interfere with MAb binding, and the MAbs do not interfere with DNA label detection. In a preferred embodiment, the label on the reporter antibody does not interfere with either the reporter MAb or capture MAb binding, and the MAbs do not interfere with label detection.

The assays of this invention are further based in one aspect on the surprising finding that forward, two-site immunoassays exhibit a linear range of detection of analyte even when the analyte is present in an amount which is orders of magnitude higher than the concentration of the reporter antibody. Previously reported ultrasensitive immunoassays commonly employed reporter antibody concentrations of between about 133 pM to 10,000 pM. See, e.g., Zhou et al., NAR (1993) 21:6038; Hendrickson et al., NAR (1995) 23:522; Joerger et al., Clin Chem (1995) 41:1371; Sims et al., Anal Biochem (2000) 281:230; Furuya et al., J Immunol Meth (2000)238:173; McKie et al., J Immunol Meth (2002) 270:135; Rissin et al., Nature Biotechnol (2010) 28:595; Rissin et al., Anal Chem (2011) 83:2279; Song et al., J Immunol Meth (2011) 372:177 (calculating antibody concentrations used in the assays reported in these references based on the assumption that the reporter antibodies used in these studies were IgG antibodies, with molecular weights of approximately 150,000 or 150 Kd). The hundred or thousand-fold excess antibody concentrations, compared with the teachings of the instant disclosure, employed in these assays would be expected to drive the binding equilibrium between the reporter antibody and the analyte to a high fraction of analyte bound to the reporter antibody, resulting in increased molar concentrations of the reporter MAb-analyte complex, and an increased signal and higher sensitivity.

The assays of the subject invention, however, are based in one aspect on the surprising observation that for forward two-site immunoassays using high affinity reporter antibodies having an equilibrium binding constant in the range of 10⁻⁸ to 10⁻¹⁰ M or smaller, the equilibrium binding constant is not the only determinative factor for assay sensitivity. Instead, it was surprisingly found that the dissociation constant (k_{d}) of the reporter antibody is a determinative factor for signal generation in a forward two-site immunoassay using a highly sensitive label, such as a DNA label detected by PCR. According to this invention, for two-site immunoassays using reporter antibodies with a threshold equilibrium binding constant of at least 1×10⁻⁸ M, the sensitivity is determined primarily by the dissociation constant for the binding of the reporter antibody to the analyte, and not by the association constant or the equilibrium binding constant of the reporter antibody. In one embodiment, reporter monoclonal antibodies having the lowest disssociation constants, or dissociation constants of less than about 3×10⁻⁴ sec⁻¹ provide greater sensitivity and more robust measurements. In other embodiments, it was surprisingly observed that reporter monoclonal antibodies having a dissociation constant of less than about 3×10⁻⁴ sec⁻¹ permitted detection of analytes at concentrations as low as 0.1 fM.

It was also surprisingly observed that the assays of this invention exhibited linearity over a range of analyte concentrations which exceeded the concentration of the reporter antibody. Thus, according to the invention described herein, the dynamic range of the immunoassays of this invention are extended by using a low concentration of reporter antibody and running the assays under conditions where only a small percent of the reporter antibody is consumed.

In one aspect, the reporter antibody used in the assays of this invention will disassociate from the analyte with a dissociation constant of about 3.0×10⁻⁴ sec⁻¹ or less. In another aspect, the reporter antibody used in the assays of this invention will disassociate from the analyte with a dissociation constant of less than about 6.0×10⁻⁵ sec⁻¹, less than about 5.9×10⁻⁵ sec⁻¹, or less than about 5.3×10⁻⁵ sec⁻¹. In one aspect, the reporter antibody used in the assays of this invention will disassociate from the analyte with a dissociation constant ranging from about 5.3×10⁻⁵ sec⁻¹ to 1.1×10⁻⁴ sec⁻¹, or with a dissociation constant falling within that range.

In one embodiment, the reporter specificity molecule or antibody may be present at a concentration ranging from about 0.1 to about 30 pM, 0.1 to about 15 pM, from about 0.1 to about 10 pM; from about 0.1 to about 1.0 pM; from about 1.0 to about 5 pM; from about 5 to 10 pM; or from about 10 to 15 pM. In some embodiments, the reporter antibody or specificity molecule may be present at a concentration, for example, of about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 16.0, 17.0, 18.0, 19.0, 20.0, 21.0, 22.0, 23.0, 24.0, 25.0, 26.0, 27.0, 28.0, 29.0, 30.0, 35.0, 40.0, 45.0, 50.0, 55.0, 60.0, 65.0, 70.0, 75.0, 80.0, 85.0, 90.0, 95.0, or of about 100.0 pM, or at about equivalent weight (ng/mL) concentrations or ranges of concentrations. For example, in other embodiments, the reporter antibody may be present at a concentration ranging from about 0.015 ng/mL to about 4.50 ng/mL; from about 0.015 ng/mL to about 2.25 ng/mL; from about 0.015 ng/mL to about 1.50 ng/mL; from about 0.015 ng/mL to about 0.15 ng/mL; from about 0.15 ng/mL to about 0.75 ng/mL; from about 0.75 to 1.5 ng/mL; or from about 1.5 to 2.25 ng/mL. In some embodiments, for example, the reporter antibody may be present at a concentration of about 0.015 ng/mL, about 0.15 ng/mL, about 0.75 ng/mL, about 1.125 ng/mL, about 1.5 ng/mL, about 1.875 ng/mL, about 2.25 ng/mL, about 2.625, about 3.0 ng/mL, about 3.75 ng/mL, about 4.5 ng/mL, about 5.25 ng/mL, about 6.0 ng/mL, about 6.75 ng/mL, about 7.5 ng/mL, about 8.25 ng/mL, about 9.0 ng/mL, about 9.75 ng/mL, about 10.5 ng/mL, about 11.25 ng/mL, about 12.0 ng/mL, about 12.75 ng/mL, about 13.5 ng/mL, about 14.25 ng/mL, or about 15.0 ng/mL.

In other aspects, this invention also relates to a method for screening antibodies for use in forward, two-site immuno PCR assays capable of detecting picomolar, femtomolar, or subfemtomolar concentrations of analytes, and kits for use in the assays.

### DESCRIPTION OF FIGURES

FIG. 1 is a graph of the fraction of total antigen bound over time as four simulated antibody+analyte reactions proceed to equilibrium for four antibodies having different equilibrium binding constants (K_{d·}), as described in Table 1 (the reporter antibody concentration is assumed to be 10 pM=1×10⁻¹¹M).

FIG. 2 is a graph of the fraction of total antigen bound over time in simulated binding reactions for four different antibodies having the same equilibrium binding constants (K_{d} of 10⁻¹⁰ M), but different association (kₐ) and dissociation constants (k_{d}), as described in Table 2, herein (the reporter antibody concentration is assumed to be 10 pM=1×10⁻¹¹M).

FIG. 3 is a graph the fraction of total antigen bound over time as four simulated antibody+analyte reactions proceed to equilibrium for 4 different commercially available antibodies specific for HIV p24 protein. The rate and equilibrium constants for the four antibodies are described in Table 3 (the reporter antibody concentration is assumed to be 10 pM=1×10⁻¹¹M).

FIG. 4 is a graph describing the predicted fraction of bound p24 antigen for the four antibodies shown in FIG. 3 under simulated assay conditions where binding of the reporter antibody to antigen takes place for 120 minutes, with a 30 minute further incubation with the capture antibody, followed by a series of washes (the reporter antibody concentration is assumed to be 10 pM=1×10⁻¹¹M).

FIG. 5 is a graph describing the same assay time course simulations based on the kinetic constants for the 4 HIV antibodies (as in FIG. 4, above) but the amount of p24 binding is expressed as the estimated number of p24 molecules expected to be bound to the antibody given an initial p24 concentration of 250 fg/mL in the sample (the reporter antibody concentration is assumed to be 10 pM=1×10⁻¹¹M).

FIG. 6 is a graph describing simulated fraction of total antigen bound over time for high affinity monoclonal antibodies with equilibrium constants of K_{d}=10⁻¹⁰M but with different association (kₐ) and dissociation constants (k_{d}), as described in Table 4. This simulation assumes reporter antibody at a concentration on the order of about 10 pM.

FIG. 7 is a graph describing the fraction of total antigen bound over time for antibodies having the same kinetic parameters of FIG. 6, if those antibodies were used in an assay for HIV. The bound fraction values are expressed in terms of molecules of antigen bound for a 10 fM (=about 250 fg/mL for HIV p24) input level of analyte.

FIG. 8 is a graph describing the signal obtained above background for an exemplary PSA assay used to detect an HIV p24 concentration of 0.1 pg/mL with varying concentrations of reporter anti-HIV p24 MAb-DNA conjugate.

FIG. 9 is a graph describing the signal obtained above background for an exemplary assay for the detection of TNF alpha at 5 pg/mL, with varying concentrations of reporter anti-TNF-alpha MAb-DNA conjugate.

FIG. 10 is a graph describing the signal obtained above background for an exemplary assay for the detection of TNF alpha at 10 pg/mL, with varying concentrations of reporter anti-TNF-alpha MAb-DNA conjugate.

FIG. 11 is a graph describing another comparison of the signals obtained above background for an exemplary assay for the detection of TNF alpha from 0.625 to 10 pg/mL using both the forward and the reverse assay formats.

FIG. 12 is a graph describing the signal obtained above background for an exemplary PSA assay used to detect a PSA concentration of 2.5 pg/mL with varying concentrations of reporter anti-PSA MAb-DNA conjugate. 0.1 pg/mL HIV p24

FIG. 13 is a standard curve for calculating HIV p24 concentration vs. threshold counts.

### DETAILED DESCRIPTION

In some embodiments, the assays of this invention are two-site assays which can detect analyte present in a sample at levels or concentrations of less than about 10 pg/mL (e.g., less than about 1 pg/mL, less than about 0.1 pg/mL, less than about 10 fg/mL, or other lower threshold amounts of analyte. The specificity molecules used in the two-site assays may be, for example, antibodies, receptors, ligands, or other molecules capable of specifically binding to the analyte. In some embodiments the assays are run in the forward orientation. In another aspect, the two-site assay is a forward immunoassay which employs two antibodies which specifically recognize the antigen to be detected, or which employs one antibody and one specificity molecule which both recognize the analyte to be detected. A “capture” specificity molecule or antibody is used to coat the surface of a solid support, such as a microtiter well, bead or particle. A “reporter” specificity molecule or antibody is labeled with a high sensitivity detection molecule, such as a nucleic acid molecule, a chemiluminescent label or a nanoparticle label. In certain embodiments, the two-site immunoassay uses a DNA molecule as the label. In other embodiments, PCR is used to detect the nucleic acid or DNA label.

Because PCR can detect single molecules of nucleic acid by exponential amplification, immuno-PCR assays offer the potential for high sensitivity. However, that potential has been limited by corresponding high background which interferes with signal detection. For example, in an immuno-PCR assay any failure to remove all of the nonspecifically bound template DNA molecules results in significant background and interferes with the ability to detect minute quantities of analyte. Background in an immuno-PCR assay can be generated from nonspecific binding of any component, including non-specific binding by the reporter antibody-DNA conjugate, or by any other secondary labeling component.

Other approaches have used reverse sandwich immunoassay formats to capture and concentrate the analyte in the initial step and/or have used high reporter antibody concentrations and/or antibodies with a high equilibrium binding constant in an attempt to drive the formation of the sandwich complex. For example, immuno-PCR assays in a reverse sandwich immunoassay format use a capture antibody coated on a solid surface to immobilize the analyte, and then further complexes the bound antigen with a reporter antibody labeled directly with a molecule of DNA. As with all heterogeneous immuno PCR assays, stringent washing of the solid support is used to reduce the background signal from nonspecifically bound label DNA. Detection limits for the reverse sandwich immuno PCR assay format can exceed those obtained in enzyme immunoassays (ELISAs) by two to three orders of magnitude; however, problems with high background have limited the sensitivity that could be achieved with immuno-PCR assays.

Although stringent, numerous or prolonged washes to reduce non-specific binding can be used to reduce the background, such washing procedures can result in reduction in signal as a result of elution of the reporter antibody-analyte complex from the solid surface, or dissociation of antigen/antibody complexes. Moreover, extensive intermediate washing steps in many reported Immuno-PCR assays can be cumbersome and lengthy. In addition, nonspecific binding of label DNA created background noise, which interfered with signal detection and limited sensitivity. The resulting amplification products produced significant contamination and gave rise to false-positives, common to all DNA amplification-based assays.

Immuno-PCR is further limited by the well-known non-linearity of PCR, which can frustrate attempts to assess the proportionate amounts of analyte present. These practical difficulties have prevented Immuno-PCR techniques from gaining widespread acceptance and utility in the field of medical diagnostics.

The assays of this invention are based on the empirical determination and surprising finding that the highest signal to noise ratio in an Immuno-PCR assay is obtained by selecting a reporter antibody based on the lowest dissociation constant rather than selecting the highest affinity antibody with the lowest equilibrium binding constant or the antibody with the highest association constant. The inventors have determined that selecting a reporter antibody with a low dissociation constant maintains the immobilized two-site immune complex intact during washing steps to maximize signal strength, while also enhancing the removal of any non-specifically bound reporter antibody, thereby minimizing background. Use of a reporter antibody with a low dissociation constant is especially advantageous when used in a forward immuno-PCR assay, as described herein.

In another embodiment, the assays of this invention are based on the additional surprising observation that use of the reporter antibody at low concentrations ranging between 0.1 pM to 10 pM, 3.0 to 5.0 pM, 5.0 to 10.0-pM, or between 3.0 pM to 10 pM, permits formation of sufficient reporter antibody-analyte complexes in a forward two-site immunoassay, to yield a higher signal to noise than use of a reporter antibody at a higher concentration such as 100 pM or higher. The concentration of the reporter antibody is the concentration of the antibody present during incubation of the reporter antibody with the analyte.

In one embodiment, the reporter antibody may be present at a concentration ranging from about 0.1 to about 30 pM, 0.1 to about 15 pM, from about 0.1 to about 10 pM; from about 0.1 to about 1.0 pM; from about 1.0 to about 5 pM; from about 5 to 10 pM; from about 10 to 15 pM, from about 3.0 to about 5.0 pM, 5, or from about 3.0 pM to about 10 pM. In some embodiments, the reporter antibody may be present at a concentration, for example, of about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 16.0, 17.0, 18.0, 19.0, 20.0, 21.0, 22.0, 23.0, 24.0, 25.0, 26.0, 27.0, 28.0, 29.0, or of about 30.0 pM, or equivalent weight concentrations or ranges based on the molecular weight of the antibody-conjugate complex and/or assuming an antibody molecular weight of 150 Kd. In other embodiments the reporter antibody may be present at a concentration, for example, of 20, 25, or 30 pM or equivalent weight concentrations. In other embodiments, the reporter antibody may be present at a concentration ranging from about 0.015 ng/mL to about 4.50 ng/mL; from about 0.015 ng/mL to about 2.25 ng/mL; from about 0.015 ng/mL to about 1.50 ng/mL; from about 0.015 ng/mL to about 0.15 ng/mL; from about 0.15 ng/mL to about 0.75 ng/mL; from about 0.75 to 1.5 ng/mL; or from about 1.5 to 2.25 ng/mL. In some embodiments, the reporter antibody may be present at a concentration of about 0.015 ng/mL, about 0.15 ng/mL, 0.75 ng/mL, about 1.125 ng/mL, about 1.5 ng/mL, about 1.875 ng/mL, or about 2.25 ng/mL.

Moreover, it has been further demonstrated that reporter antibody concentration is not a limiting factor in achieving linearity over a range of analyte concentrations which exceeds the reporter antibody concentration. Although the use of low quantities of binding agent has been suggested for use in an array format assay, see U.S. Pat. No. 5,432,099 and U.S. Pat. No. 5,599,720, the inventors herein believe that they are the first to demonstrate that concentrations of antibodies ranging from between 0.1 pM to 10 pM or 3.0 pM to 10 pM surprisingly yield a higher sensitivity in a forward immuno-PCR assay than the sensitivity obtained when using a reporter antibody at a higher concentration such as 100 pM.

In one embodiment this invention relates to a two-site immunoassay for detecting analytes present in a sample at concentrations of less than about 10 pg/mL, or other lower threshold amounts of analyte. In certain embodiments, the two-site immunoassay of this invention is a forward two-site immunoassay which employs two antibodies which specifically recognize the antigen to be detected. A “reporter” antibody is labeled with a high sensitivity detection molecule, such as a nucleic acid molecule, a chemiluminescent label or a nanoparticle label. A “capture” antibody is also used to coat the surface of a solid support, such as a microtiter well, bead or particle. In certain embodiments, the two-site immunoassay uses monoclonal antibodies and a DNA molecule as the label.

In one embodiment, this invention provides a method for detecting a non-nucleic acid analyte present in a sample to be tested using a forward two-site immunoassay, the method comprising the steps of:
(1) contacting the sample containing the analyte with:
   (a) a reporter conjugate present at a concentration ranging from about 1 to 15 pM or from about 0.15 to 2.25 ng/mL of antibody protein, wherein the reporter conjugate comprises:
      (i) a reporter monoclonal antibody capable of specifically binding the analyte in the test sample; and
      (ii) a label;

thereby forming a first immune complex; and
(2) contacting the first immune complex with a capture monoclonal antibody bound to a solid support, where the capture antibody is capable of specifically binding to the analyte in the first immune complex, thereby forming a two-site immune complex bound to the support;
(3) washing the two-site immune complex bound to the support; and
(4) detecting the presence and amount of the analyte, and wherein the assay detects analyte present at concentrations of less than about 10 picogram/mL.

In some embodiments the concentration of antibody may be either lower or higher than 15 pM and/or range either lower or higher than 15 pM, as discussed herein. In addition, as discussed herein, in some embodiments, the concentration of analyte detected may be present at concentrations lower than 10 pg/mL, as discussed herein.

In another embodiment, this invention provides a method for detecting a non-nucleic acid analyte present in a sample to be tested using a forward two-site immunoassay, the method comprising the steps of:
(1) contacting the sample containing the analyte with:
   (b) a reporter conjugate present at a concentration ranging from about 1 to 15 pM or from about 0.15 to 2.25 ng/mL of antibody protein, wherein the reporter conjugate comprises:
      (i) a reporter monoclonal antibody capable of specifically binding the analyte in the test sample with a dissociation rate constant lower than about 3.0×10⁻⁴ sec⁻¹; and
      (ii) a label;

thereby forming a first immune complex; and
(2) contacting the first immune complex with a capture monoclonal antibody bound to a solid support, where the capture antibody is capable of specifically binding to the analyte in the first immune complex, thereby forming a two-site immune complex bound to the support;
(3) washing the two-site immune complex bound to the support; and
(4) detecting the presence and amount of the analyte, and wherein the assay detects analyte present at concentrations of less than about 10 picogram/ml.

In some embodiments the concentration of antibody may be either lower than 1 pM or higher than 15 pM and/or range either lower than 1 pM or higher than 15 pM, as discussed herein. In addition, as discussed herein, in some embodiments, the concentration of analyte detected may be lower than 10 pg/mL, as discussed herein. Moreover, the dissociation rate constant of the reporter antibody may be at a rate lower than 1.1×10⁻⁴ sec⁻¹, less than 5.9×10⁻⁵ sec⁻¹, or any other rate or range discussed herein.

The reporter monoclonal antibody in the reporter conjugate may be labeled directly by covalent modification of the antibody or indirectly through the binding of a second antibody or protein possessing a detectable label. Indirect attachment may be accomplished, for example, by labeling the reporter antibody with biotin either directly or through a linker and attaching the detection molecule to avidin, or any other high affinity binding pair. The label may be a nucleic acid label, and detection of the label may be carried out by nucleic amplification. For example, label detection may be carried out using PCR, and amplicon generation may in some instances be monitored by real-time PCR.

The capture antibody is specific for a second site on the analyte with an epitope different from the epitope to which the reporter antibody binds. The capture antibody may be a monoclonal antibody. In one embodiment the capture MAb is coated onto a solid surface, which, in some embodiments will be paramagnetic microparticles suitable for use in automated assays.

In some embodiments, the solid support to which the immobilized two-site immune complex is bound is washed several times to remove excess reporter antibody-label conjugate. In one embodiment the solid support is paramagnetic microparticles. The solid support may also be, for example, latex or other polymer beads, glass beads or fibers, nanoparticles, insoluble polysaccharides, for example, dextran, or other solid phase supports well known in the art.

When paramagnetic microparticles are used as a solid support, the microparticles to which the immobilized sandwich immune complexes are bound are washed by several cycles of magnetic capture and re-suspension in order to remove excess reporter antibody-label conjugate. In some embodiments the washing procedure may be carried out using instrumentation, and the instrumentation may be capable in some embodiments of automation of one or more steps.

In one aspect of this invention, the sample containing the two-site immune complex is subjected to signal generating conditions in order to detect two-site immune complexes which are specifically bound through binding between an attached reporter antibody and the analyte. For example, where the label is a PCR label, the two-site immune complex is subjected to PCR conditions, which include contact with reagents and primers for conducting a polymerase chain reaction, and conditions for generating amplicons. The generation of amplicons is monitored by PCR. In one embodiment the monitoring of the PCR reaction may be performed in real time.

Analyte can be quantitated by monitoring the number of PCR thermocycles, or the time that it takes to generate a predetermined fluorescent signal over baseline. Analyte quantitation can also be accomplished by any method for monitoring amplicon production, and/or any method or algorithm known in the art for analyzing and quantitating the production of amplicons. For example, in one aspect this is accomplished by automatic instrumentation designed to monitor fluorescence intensity as a function of cycle number. When using threshold cycle counts, the amount of DNA initially present in the sample is inversely proportional to the threshold cycle (Ct). antigen concentrations (pg/mL) of the samples are calculated from the calibration curve.

In one aspect, the reporter antibody used in the assays of this invention will disassociate from the analyte with a dissociation constant of less than about 3.0×10⁻⁴ sec⁻¹ or 1.1×10⁻⁴ sec⁻¹. In another aspect, the reporter antibody used in the assays of this invention will disassociate from the analyte with a dissociation constant of less than about 6.0×10⁻⁵ sec⁻¹, or less than about 5.9×10⁻⁵ sec⁻¹. In one aspect, the reporter antibody used in the assays of this invention will disassociate from the analyte with a dissociation constant ranging from about 5.5×10⁻⁵ to 3.0×10⁻⁴ sec⁻¹. In another aspect, the reporter antibody used in the assays of this invention will disassociate from the analyte with a dissociation constant ranging from about 5.3×10⁻⁵ to 3.0×10⁻⁴ sec⁻¹. The reporter antibody used in the assays if this invention may disassociate from the analyte with a dissociation constant of less than about 5.3×10⁻⁵, 5.4×10⁻⁵, 5.5×10⁻⁵, 5.6×10⁻⁵, 5.7×10⁻⁵, 5.8×10⁻⁵, 5.9×10⁻⁵, 6.0×10⁻⁵, 6.1×10⁻⁵, 6.2×10⁻⁵, 6.3×10⁻⁵, 6.4×10⁻⁵, 6.5×10⁻⁵, 6.6×10⁻⁵, 6.7×10⁻⁵, 6.8×10⁻⁵, 6.9×10⁻⁵, 7.0×10⁻⁵, 7.1×10⁻⁵, 7.2×10⁻⁵, 7.3×10⁻⁵, 7.4×10⁻⁵, 7.5×10⁻⁵, 7.6×10⁻⁵, 7.7×10⁻⁵, 7.8×10⁻⁵, 7.9×10⁻⁵, 8.0×10⁻⁵, 8.1×10⁻⁵, 8.2×10⁻⁵, 8.3×10⁻⁵, 8.4×10⁻⁵, 8.5×10⁻⁵, 8.6×10⁻⁵, 8.7×10⁻⁵, 8.8×10⁻⁵, 8.9×10⁻⁵, 9.0×10⁻⁵, 9.1×10⁻⁵, 9.2×10⁻⁵, 9.3×10⁻⁵, 9.4×10⁻⁵, 9.5×10⁻⁵, 9.6×10⁻⁵, 9.7×10⁻⁵, 9.8×10⁻⁵, 9.9×10⁻⁵, 1.0×10⁻⁴, 1.1×10⁻⁴, 1.2×10⁻⁴, 1.3×10⁻⁴, 1.4×10⁻⁴, 1.5×10⁻⁴, 1.6×10⁻⁴, 1.7×10⁻⁴, 1.8×10⁴, or 1.9×10⁻⁴, 2.0×10⁻⁴, 2.1×10⁻⁴, 2.2×10⁻⁴, 2.3×10⁻⁴, 2.4×10⁻⁴, 2.5×10⁻⁴, 2.6×10⁻⁴, 2.7×10⁻⁴, 2.8×10⁻⁴, 2.9×10⁻⁴, 3.0×10⁴. 3.1×10⁻⁴, 3.2×10⁻⁴, 3.3×10⁻⁴, 3.4×10⁻⁴, 3.5×10⁻⁴, 3.6×10⁻⁴, 3.7×10⁻⁴, 3.8×10⁻⁴, 3.9×10⁻⁴ or about 4.0×10⁻⁴ sec⁻¹.

In one embodiment, the reporter antibody may be present at a concentration ranging from about 0.1 to about 30 pM, 0.1 to about 15 pM, from about 0.1 to about 10 pM; from about 0.1 to about 1.0 pM; from about 1.0 to about 5 pM; from about 5 to 10 pM; from about 10 to 15 pM, from about 3.0 to about 5.0 pM, 5, or from about 3.0 pM to about 10 pM. In some embodiments, the reporter antibody may be present at a concentration, for example, of about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5 or of about 15 pM. The reporter antibody concentration may also be expressed as a weight concentration equivalent to the molar concentrions of the reporter antibodies of this invention, based on the molecular weight of the antibody-conjugate complex and/or assuming a molecular weight for the labeled reporter antibody of 150 Kd.

In another aspect, the assays of this invention may be used to detect analytes present at concentrations of about 0.01, 0.02, 0.03, 0.04, 0.05, 0.06, 0.07, 0.08, 0.09, 0.1, 0.125, 0.150, 0.175, 0.2, 0.225, 0.250, 0.175, 0.3, 0.325, 0.350, 0.375, 0.400, 0.4, 0.425, 0.450, 0.475, 0.5, 0.525, 0.550, 0.575 0.6, 0.625, 0.650, 0.675, 0.7, 0.725, 0.750, 0.775, 0.8, 0/825, 0.850, 0.875, 0.9, 0.925, 0.950, 0.975, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9 or 15.0 pg/mL or equivalent pM concentrations.

In other aspects, the assays of this invention may have limits of detection of about about 0.01, 0.02, 0.03, 0.04, 0.05, 0.06, 0.07, 0.08, 0.09, 0.1, 0.125, 0.150, 0.175, 0.2, 0.225, 0.250, 0.175, 0.3, 0.325, 0.350, 0.375, 0.400, 0.4, 0.425, 0.450, 0.475, 0.5, 0.525, 0.550, 0.575 0.6, 0.625, 0.650, 0.675, 0.7, 0.725, 0.750, 0.775, 0.8, 0/825, 0.850, 0.875, 0.9, 0.925, 0.950, 0.975, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9 or 15.0 pg/mL or equivalent pM concentrations.

In one embodiment, the reporter antibody may be present at a concentration ranging from about 0.1 to about 15 pM, from about 0.1 to about 10 pM; from about 0.1 to about 1.0 pM; from about 1.0 to about 5 pM; from about 5 to 10 pM; or from about 10 to 15 pM. In some embodiments, the reporter antibody may be present at a concentration, for example, of about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15, 20, 25, 30 pM, 40 pM, 50 pM, 60 pM, 70 pM, 80 pM, 90 pM, or of about 100 pM, or equivalent weight concentrations or ranges of concentrations. The reporter antibody concentration may also be expressed as a weight concentration equivalent to the molar concentrions of the reporter antibodies of this invention, based on the molecular weight of the antibody-conjugate complex and/or assuming a molecular weight for the labeled reporter antibody of 150 Kd. For example, in other embodiments, the reporter antibody may be present at a concentration ranging from about 0.015 ng/mL to about 4.50 ng/mL; from about 0.015 ng/mL to about 2.25 ng/mL; from about 0.015 ng/mL to about 1.50 ng/mL; from about about 0.015 ng/mL to about 0.15 ng/mL; from about 0.15 ng/mL to about 0.75 ng/mL; from about 0.75 to 1.5 ng/mL; or from about 1.5 to 2.25 ng/mL. In some embodiments, for example, the reporter antibody may be present at a concentration of about 0.015 ng/mL, about 0.15 ng/mL, about 0.75 ng/mL, about 1.125 ng/mL, about 1.5 ng/mL, about 1.875 ng/mL, about 2.25 ng/mL, about 2.625, about 3.0 ng/mL, about 3.75 ng/mL, about 4.5 ng/mL, about 5.25 ng/mL, about 6.0 ng/mL, about 6.75 ng/mL, about 7.5 ng/mL, about 8.25 ng/mL, about 9.0 ng/mL, about 9.75 ng/mL, about 10.5 ng/mL, about 11.25 ng/mL, about 12.0 ng/mL, about 12.75 ng/mL, about 13.5 ng/mL, about 14.25 ng/mL, or about 15.0 ng/mL.

Exemplary reporter conjugate concentrations further include concentration ranges of, for example, about 3, 4 or about 5 pM to about 15 pM, including, for example, about 6 to about 14 pM, about 7 to about 13 pM, about 8 to about 12 pM, about 9 pM to about 11 pM, and about 10 pM. In certain other embodiments, the reporter conjugate concentration can range from about 6 to about 15 pM, about 7 to about 15 pM, about 8 to about 15 pM, about 9 to about 15 pM, about 10 to about 15 pM, about 11 to about 15 pM, about 12 to about 15 pM, about 13 to about 15 pM and about 14 to about 15 pM. In certain other embodiments, the reporter conjugate concentration can range from about 5 to about 14 pM, about 5 to about 13 pM, about 5 to about 12 pM, about 5 to about 11 pM, about 5 to about 10 pM, about 5 to about 9 pM, about 5 to about 8 pM, about 5 to about 7 pM, about 5 to about 6 pM. In certain other embodiments, the reporter conjugate concentration can range from about 6 to about 10, from about 7 to about 10, from about 8 to about 10 or from about 9 to about 10 pM. In certain embodiments, the reporter conjugate concentration can range from about 4 to about 14 pM, about 4 to about 13 pM, about 4 to about 12 pM, about 4 to about 11 pM, about 4 to about 10 pM, about 4 to about 9 pM, about 4 to about 8 pM, about 4 to about 7 pM, or from about 4 to about 6 pM. In certain embodiments, the reporter conjugate concentration can range from about 3 to about 14 pM, about 3 to about 13 pM, about 3 to about 12 pM, about 3 to about 11 pM, about 3 to about 10 pM, about 3 to about 9 pM, about 3 to about 8 pM, about 3 to about 7 pM, or about 3 to about 6 pM. Equivalent weight ranges of reporter antibody may also be used.

In some aspects, the reporter conjugate is contacted with the sample containing the analyte for about 90-150 minutes, for about 100-120 minutes, or for about 90, 95, 100, 105, 110, 115, or about 120 minutes.

In one aspect, the reporter-conjugate-analyte complex is contacted with the capture antibody for about 20-60 minutes, for about 30-60 minutes, for about 30-45 minutes, or for about 20, 25, 30, 35, 40, 45, 50, 55, or 60 minutes.

In another aspect, the sample containing the solid support and the two-site immune complex is washed for about 30, 35, 40, 45, 50, 55, 60, 65, 70, 75, 80, 85, 90, 95, 100, 105, 110, 115, or about 120 minutes. In other aspects, the sample containing the solid support and the two-site immune complex is washed 2, 3, 4, 5, 6, 7, 8, 9, or 10 times.

In other embodiments, this invention relates to methods for screening antibodies for use as a reporter antibody in a forward, two-site immuno-PCR assay, the method comprising
(1) obtaining the dissociation constant for two or more antibodies;
(2) performing the forward two-site immunoassays of this invention with at least the following two antibodies used as reporter antibodies in each assay:
   (c) an antibody with a k_{d} value of less than about 3×10⁻⁴ sec⁻¹; and
   (d) an antibody a k_{d} value greater than about 3×10⁻⁴ sec⁻¹; and
(3) determining that the methods of the invention performed with the screened antibody with a k_{d} value of less than about 3×10⁻⁴ sec⁻¹ has a higher sensitivity or other advantageous characteristics, such as greater detectable signal above background, higher linearity, or higher dynamic range, than the methods of the invention performed with the screened antibody with a k_{d} value greater than 3×10⁻⁴ sec⁻¹.

The screening method may also be performed on one or more or each of the antibodies for which dissociation constants are obtained, to confirm that antibodies having a k_{d} value of less than about 3×10⁻⁴ sec⁻¹ or in the range of about 3×10⁻⁴ sec⁻¹ to 5×10⁻⁵ sec⁻¹ provide higher sensitivities and more robust results than assays performed with antibodies having higher k_{d} values. The screening method may also be performed with any other subset of antibodies for which k_{d} values are obtained. As an example, the method may be performed using the antibody with the lowest k_{d} value and the antibody with the second lowest k_{d} value to determine if the antibody with the lowest k_{d} value yields the assay with the highest sensitivity. However, for antibodies having a k_{d} value of less than about 3×10⁻⁴ sec⁻¹, other factors that affect immunoassay performance may also determine which screened antibody provides the highest sensitivity. Those factors include the tendency of an antibody to non-specifically stick to the solid substrate or other components of the assay, interaction between the receptor antibody and the capture antibody when bound or free, or other factors,

The equilibrium binding constant, dissociation constant and association constant for the reporter antibody may be determined by methods known to those of ordinary skill in the art, such as with a Biocore system (GE Healthcare) or a KinExA® (Kinetic Exclusion Assay) assay, available from Sapidyne Instruments (Boise, Id.). The equilibrium binding constant, dissociation constant and/or association constant for the reporter antibodies may be obtained by measuring the constants using any known method, or by obtaining test results.

Where the equilibrium binding constant, dissociation constant and association constant are obtained for three or more antibodies, two-site immunoassays are performed, each using as a reporter antibody at least the antibody with the lowest dissociation constant, and the antibody with the highest equilibrium constant or the highest association constant. If the antibody with the lowest dissociation constant is also the antibody with the lowest equilibrium binding constant (i.e., highest affinity), then the antibody with the second highest equilibrium binding constant or the antibody with the highest association constant can be used for comparison of assay sensitivity using two or more antibodies.

In other embodiments, this invention relates to a kit for detecting a non-nucleic acid analyte, which comprises:
(1) a first container comprising a reporter monoclonal capable of specifically binding to an analyte and having a dissociation rate constant lower than about 3.0×10⁻⁴ sec⁻¹, wherein the reporter monoclonal antibody is attached to an assay specific DNA label;
(2) a second container containing a capture monoclonal antibody for the analyte; wherein the capture monoclonal antibody is attached to a solid support.
   The kit may also come with instructions and/or provide reporter antibody at a concentration such that the concentration of the reporter antibody during the incubation with analyte in the sample will be present at a low concentration as described herein.

Detecting extremely small amounts of an analyte can be important for determining disease states, screening for diseases or medical conditions, identifying exposure to or reveal the presence or absence of pollutants, carcinogens, allergens, radiation, toxins, contaminants, infectious agents, and drugs.

While the present invention has been described in conjunction with specific embodiments set forth above, many alternatives, modifications and variations thereof will be apparent to those of ordinary skill in the art. All such alternatives, modifications and variations are intended to fall within the spirit and scope of the present invention.

### DEFINITIONS

Unless specific definitions are provided, the nomenclature utilized in connection with, and the laboratory procedures, techniques and methods described herein are those known in the art to which they pertain. Standard chemical symbols and abbreviations are used interchangeably with the full names represented by such symbols. Thus, for example, the terms “hydrogen” and “H” are understood to have identical meaning Standard techniques may be used for chemical syntheses, chemical analyses, pharmaceutical preparation, formulation, delivery, and treatment of patients. Standard techniques may be used for recombinant DNA methodology, oligonucleotide synthesis, tissue culture and the like. Reactions and purification techniques may be performed, e.g., using kits according to manufacturer's specifications, as commonly accomplished in the art or as described herein. The foregoing techniques and procedures may be generally performed according to conventional methods well known in the art and as described in various general or more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), Harlow & Lane, Antibodies: A Laboratory Manual (Cold Spring Harbor Laboratory Press, Cold Spring Harbor, N.Y. (1988)), which are incorporated herein by reference in their entirety for any purpose.

As used herein, the term “sample” can include both processed and unprocessed samples to be tested, but may also refer to the mixture comprising the analyte and/or immune complexes comprising the analyte at various stage of the immunoassay. In certain embodiments, samples may also comprise test samples containing components with two interaction sites suitable for an immunoassay. Exemplary test samples may include an aliquot of material, frequently an aqueous solution or an aqueous suspension derived from biological source. Exemplary samples to be assayed for the presence of an analyte by the methods of the present disclosure can include, for example, cells, tissues, homogenates, lysates, extracts, purified or partially purified proteins and other biological molecules and mixtures thereof. Non-limiting examples of samples typically used in the methods of the disclosure include human and animal body fluids such as whole blood, serum, plasma, cerebrospinal fluid, sputum, bronchial washings, bronchial aspirates, urine, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; and cell specimens which may or may not be fixed. Samples also include fetal analytes or cells present in maternal samples.

The samples used in the methods of the present invention will vary based on the assay format and the nature of the tissues, cells, extracts or other materials, especially biological materials, to be assayed. Methods for preparing, for example, homogenates and extracts, such as protein extracts, from cells or other samples are well known in the art and can be readily adapted in order to obtain a sample that is compatible with the methods of the invention. Methods for removing substances which might interfere with the assay are also known.

“Reporter molecule-label conjugate” as used herein, refers to a conjugate formed between a specificity molecule and a label.” The reporter molecule may be a reporter antibody.

The term “analyte,” as used herein, refers to any substance that it is desirable to detect in an assay, and which may be present in a test sample. The analyte can be, without limitation, any substance detectable by a two-site immunoassay. In certain embodiments, an analyte comprises a substance for which there exists a naturally occurring antibody or for which an antibody can be prepared. The analyte may be, for example, a protein, a polypeptide, a hapten, a carbohydrate, a lipid, a drug or drug metabolite, an infectious agent, a cell or any other of a wide variety of biological or non-biological molecules, complexes or combinations thereof. In another embodiment, the analyte is a nucleic acid. In still another embodiment the analyte is an antibody. In yet another embodiment, the analyte is a cell (animal, plant, fungal, bacterial, etc.) or a subcomponent or organelle (e.g., mitochondria) thereof.

The assays of this invention are useful to detect the presence and amount of any analyte which can be detected by a two-site immunoassay. In certain embodiments, the analyte of the present disclosure are useful for the detection of P24 HIV antibody, TNF alpha, and Prostate specific antigen (PSA)

Polyvalent ligand analytes that can be detected using compositions, methods and kits of the present invention will normally be poly(amino acids), i.e., polypeptides, proteins, polysaccharides, nucleic acids and combinations thereof. Such combinations include components of cells, tissues, bacteria, viruses, cell walls, cell membranes, cellular organelles, chromosomes, genes, mitochondria, nuclei and the like. Any nucleic acid present in an analyte can not interfere or cause background signal in nucleic acid marker formats. According to one aspect of the invention, analytes do not contain nucleic acid.

A wide variety of antigen analytes may be advantageously detected using the methods of the present invention. Such analytes can be classified according to family, with each family having similar structural features, biological functions, relationship to specific microorganisms (particularly disease causing microorganisms), and the like. Analytes may be, for example, proteins. Protein families of particular interest for the present invention include, for example, immunoglobulins, cytokines, enzymes, hormones, cancer antigens, nutritional markers, metabolic markers, tissue specific antigens, and biowarfare agents, and other analytes indicative of a disease or condition, such as cardiovascular conditions, alzheimer's disease, or other diseases or conditions for which markers are known or can be determined. These analytes may be present in blood, serum, plasma, spinal fluid, synovial fluid, saliva, urine, cells or tissues.

In addition, it may be desirable to detect the normal or diseased tissue or cells of a patient. The presence or absence of certain circulating cancer cells or other cells, or cells such as fetal cells circulating in the maternal system, for example, may be diagnostic for disease. Thus, the endogenous cells of a human patient are analytes that may be advantageously detected using the compositions, methods and kits of the present invention.

Specificity molecules are molecules which can specifically bind to an analyte.

The term “nucleic acid marker” refers to a nucleic acid molecule that will produce a detection product of a predicted size or other selected characteristic when used with appropriately designed oligonucleotide primers in a nucleic acid amplification reaction, such as a PCR reaction. Skilled artisans will be familiar with the design of suitable oligonucleotide primers for PCR and programs are available commercially and over the Internet to facilitate this aspect of the invention (see, for example, the http site: bibiserv.techfak.unibielefeld.de/genefisher). A nucleic acid marker may be linear or circular. In preferred embodiments, the nucleic acid marker will comprise a predetermined, linear nucleic acid sequence with binding sites for selected primers located at or near each end. In a circular DNA nucleic acid molecule, the primers will be internal rather than at an end, and a single primer may be used, e.g. for Rolling Circle Amplification. Amplified DNA may be detected using any available method, including, but not limited to techniques such as real-time PCR, SYBR® Green staining, or ethidium bromide staining. In other embodiments of the invention, the binding sites for the amplification primers flank an undefined DNA sequence of defined length, or a DNA sequence that comprises another identifiable characteristic, such as a detectable sequence, in addition to undefined sequences. In some embodiments, the nucleic acid marker is distinguished by the size or mass of the amplified sequences; thus, the DNA sequence between the primers need not be defined as to the exact sequence, just as to the number of bases. Alternatively, the size and/or sequence of the entire nucleic acid marker need not be defined as long as a binding site for a molecular beacon (see, infra) is supplied. In further embodiments, the DNA sequence located between the primer binding sites comprises a “characteristic identification sequence” capable of being detected during the PCR reaction. Fluorescent signal generation may, for example, be sequence-specific (Molecular Beacons, TaqMan®, fluorogenic primers, such as the LUX™ primers (Invitrogen (Carlsbad, Calif.)) or mass dependent (e.g., SYBR® Green, Ethidium Bromide). The examples provided are not meant to be an exhaustive list of possible nucleic acid detection schemes as those skilled in the art will be aware of alternative markers suitable for use in the methods of the present invention.

“Polyclonal Antibodies” or “PAbs,” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or hapten-carrier conjugate, optionally supplemented with adjuvants. Polyclonal antibodies may be unpurified, purified or partially purified from other species in an antiserum. Techniques for the preparation and purification of polyclonal antibodies are well-known in the art and are described in various general and more specific references, including but not limited to Kabat & Mayer, Experimental Immunochemistry, 2d ed., (Thomas, Springfield, Ill. (1961)); Harlow & Lane, Antibodies: A Laboratory Manual (Cold Spring Harbor Laboratory Press, Cold Spring Harbor, N.Y. (1988)); and Weir, Handbook of Experimental Immunology, 5th ed. (Blackwell Science, Cambridge, Mass. (1996)).

“Monoclonal antibodies,” or “MAbs,” which are homogeneous populations of antibodies to a particular antigen, may be obtained by any technique that provides for the production of antibody molecules, such as by continuous culture of cell lines. These techniques include, but are not limited to the hybridoma technique of Koehler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Monoclonal antibodies may also be engineered, chimerized, optimized and/or humanized by techniques known to those of skill in the art or developed in the future, such as phage display or yeast display or use of a transgenic animal system. The term monoclonal antibody may also include monoclonal antibody fragments or constructs made from those fragments, as long as the fragment or construct can specifically bind the antigen with a similar or higher affinity as the monoclonal antibody from which the fragment or construct was derived.

Antibody fragments that recognize specific epitopes may be generated by known techniques. For example, such fragments include but are not limited to: the F(ab′)2 fragments that can be produced by pepsin digestion of the antibody molecule and the Fab fragments that can be generated by reducing the disulfide bridges of the F(ab′)2 fragments. Alternatively, Fab expression libraries may be constructed (Huse, et al., Science, 246:1275-81 (1989)) to allow rapid and easy identification of monoclonal Fab fragments with the desired specificity.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies suitable for use in the present invention. Single chain antibodies are typically formed by linking the heavy and light chain fragments of the Fv region via an amino acid bridge, resulting in a single chain polypeptide.

The antibodies used in this invention may be of any immunoglobulin class including IgG, IgM, IgE, IgA, IgD and any subclass thereof. The hybridoma producing the MAb of this invention may be cultivated in vitro or in vivo. Production of high titers of MAbs in vivo makes this a presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody can be a molecule in which different portions are derived from different animal species, such as those having a variable region derived from a murine MAb and a human immunoglobulin constant region.

The term “hapten” as used herein, refers to a small proteinaceous or non-protein antigenic determinant which is capable of being recognized by an antibody. Typically, haptens do not elicit antibody formation in an animal unless part of a larger species. For example, small peptide haptens are frequently coupled to a carrier protein, such as keyhole limpet hemocyanin, in order to generate an anti-hapten antibody response. “Antigens” are macromolecules capable of generating an antibody response in an animal and being recognized by the resulting antibody. Both antigens and haptens comprise at least one antigenic determinant or “epitope,” which is the region of the antigen or hapten which binds to the antibody. Typically, the epitope on a hapten is the entire molecule.

The terms “two site antibodies” or “sandwich pair antibodies” or “sandwich antibody pairs,” as used herein, refers to a pair of typically monospecific antibodies, e.g. monoclonal antibodies, that are suitable for use in a sandwich format immunoassay. Each antibody of the pair binds to a different epitope on the same molecule and both antibodies of the pair can bind to the antigen at the same time. Methods for identifying pairs of antibodies suitable for sandwich assays will be well known to those in the art. The skilled artisan will also recognize that various other molecules can be used as sandwich pairs. For example, a receptor analyte can be sandwiched between a ligand for that receptor and an antibody that binds to an epitope on the receptor that is not involved in ligand binding. Thus, an antibody and ligand can be used as a sandwich pair in a two site assay for a receptor analyte.

As used herein, the instant disclosure also provides a two-site binding pair having a first binding member comprising a first specificity molecule coupled to a first nucleic acid, and a second binding member comprising a second specificity molecule coupled to a second nucleic acid, where the first and second nucleic acids form a two-site complex.

In certain embodiments, the reporter and capture specificity molecules may be receptors, ligands, or antibodies. The specificity molecules may be identical or different from each other. In one embodiment, the specificity molecules of the present invention interact with two receptors on a single cell. In another embodiment, both the reporter and capture specificity molecules are monoclonal antibodies, which may interact with different epitopes on the same antigen and thereby comprise a sandwich pair.

The nucleic acid markers of this invention are typically single-strand nucleic acids which may be DNA, RNA, or PNA, but may be partially double-stranded nucleic acids or analogues thereof. In certain embodiments of the invention, at least one of the nucleic acids is a chimeric DNA/RNA molecule. The nucleic acids of the invention may be coupled via their 5′ ends or their 3′ ends. In some embodiments, the nucleic acid is suitable for amplification by PCR, LCR, SDA, or TMA.

“Receptor” or “biological receptor” typically refers to a molecular structure within or on the surface a cell characterized by selective binding of a specific substance (e.g., a “ligand”) and resulting in a specific physiologic effect that accompanies the binding. Examples of receptors include cell surface receptors for peptide hormones, neurotransmitters, antigens, complement fragments, immunoglobulins and cytoplasmic receptors for steroid hormones. As used herein, however, the receptor will typically be isolated and purified and need not effect or be capable of effecting a physiological or other biological effect. The methods of the present invention may exploit the selective binding of the receptor to the specific substance by using a receptor for a ligand analyte as a capture or reporter specificity molecule. The receptors and/or ligands used as capture or reporter specificity molecules should have equilibrium binding constants of between about 10⁻¹¹ M to 10⁻⁸ M

The term “ligand” refers generally to a molecule that binds to a receptor. Typically, a ligand is a small, soluble molecule, such as a hormone or neurotransmitter.

The term “solid support” refers to any solid phase that can be used to immobilize e.g., an analyte, an antibody or a complex. Suitable solid supports will be well known in the art and include the walls of wells of a reaction tray, such as a microtiter plate, the walls of test tubes, polystyrene beads, paramagnetic or non-magnetic beads, nitrocellulose membranes, nylon membranes, microparticles such as latex particles, and sheep (or other animal) red blood cells. Typical materials for solid supports include, but are not limited to, polyvinyl chloride (PVC), polystyrene, cellulose, nylon, latex and derivatives thereof. Further, the solid support may be coated, derivatized or otherwise modified to promote adhesion of the desired molecules (e.g., analytes) and/or to deter non-specific binding or other undesired interactions. The choice of a specific “solid phase” is usually not critical and can be selected by one skilled in the art depending on the assay employed. Thus, latex particles, microparticles, paramagnetic or non-magnetic beads, membranes, plastic tubes, walls of microtiter wells, glass or silicon chips, and red blood cells are all suitable sold supports. Conveniently, the solid support can be selected to accommodate various detection methods. For example, 96 or 384 well plates can be used for assays that will be automated, for example by robotic workstations, and/or those that will be detected using, for example, a plate reader. According to one embodiment of the invention in which sandwich immunoassays are performed, the walls of the wells of a reaction tray are typically employed. In alternative embodiments of the instant invention, paramagnetic beads may be used as a solid support. Suitable methods for immobilizing molecules on solid phases include ionic, hydrophobic, covalent interactions and the like, and combinations thereof. However, the method of immobilization is not typically important, and may involve uncharacterized adsorption mechanisms. A solid support as used herein, may thus refer to any material that is insoluble, or can be made insoluble by a subsequent reaction. The solid support can be chosen for its intrinsic ability to attract and immobilize a capture reagent. Alternatively, the solid phase can retain an additional receptor that has the ability to attract and immobilize a capture reagent. The additional receptor may include a substance that is oppositely charged with respect to either the capture reagent itself or to a charged substance conjugated to the capture reagent. In yet another embodiment of the invention, an additional receptor molecule can be any specific binding member that is immobilized upon (attached to) the solid phase and which has the ability to immobilize a capture reagent through a specific binding reaction. The additional receptor molecule enables indirect immobilization of the capture reagent to a solid phase before or during the performance of the assay. The solid phase thus can be a plastic, derivatized plastic, paramagnetic or non-magnetic metal, glass or silicon surface of a test tube, microtiter well, sheet, bead, microparticle, chip, or other configurations known to those of ordinary skill in the art.

As used herein, the term “k_{assoc}” or “kₐ” or “k₁” or “k1”, as used interchangeably herein, is intended to refer to the association rate of a particular antibody-antigen interaction, whereas the term “k_{dis}” or “k_{d},” or “k₂” or “k2”, as used herein, is intended to refer to the dissociation rate of a particular antibody-antigen interaction. The term “K_{D}” or “Kd” or “K_{d}”, as used herein, is intended to refer to the equilibrium dissociation constant, which is obtained from the ratio of k_{d} to kₐ (i.e. k_{d}/kₐ) and is expressed as a molar concentration (M). K_{d} values for antibodies can be determined using methods well established in the art. A method for determining the K_{d} of an antibody is by using surface plasmon resonance, or using a biosensor system such as a Biacore® system (GE Healthcare), or a KinExA® (Kinetic Exclusion Assay) assay, available from Sapidyne Instruments (Boise, Id.) can also be used.

As used herein, the term “high affinity” for a specificity molecule or an antibody, such as an IgG antibody, refers to an antibody having a K_{d} of 10⁻⁸ M or less, 10⁻⁹ M or less, or 10⁻¹⁰ M or less for a target analyte or antigen. The high affinity specificity molecule or antibody may be a specificity molecule or an antibody with a K_{d} of between about 10⁻¹¹ M to 10⁻⁸ M.

Two performance characteristics define the lower end of an assay's ability to detect analyte near the threshold for measurement. Levels of analyte may be referred to in terms of molar concentration, weight per volume, or number of molecules present in a sample. The “limit of detection” (LOD) is the smallest amount of analyte that a method can reliably detect to determine the presence or absence of the analyte in a sample. In one aspect, the LOD is the lowest amount of analyte in a sample that can be detected with a stated probability. The LOD may also be referred to as the “lower limit of detection”, and may sometimes be used to indicate “sensitivity.” The “limit of quantitation” (LOQ) is the smallest amount the method can reliably measure quantitatively. In one aspect, the LOQ is the lowest amount of analyte in a sample that can be quantitatively determined with a stated acceptable precision, under stated experimental conditions.

The “limit of blank” (LoB) is the highest value expected to be observed in a series of results on a sample that contains no analyte. The LoB refers to an observed test result, while all of the other limits refer to actual levels of the analyte.

Procedures known to those of ordinary skill in the art may be used to determine the lower limits of detection and quantitation (LoD and LoQ) for the immunoassays of this invention. See, e.g., NCCLS publication, “Protocols for Determination of Limits of Detection and Limits of Quantitation; Approved Guideline,” EPI7-A, Vol. 24 No. 34.

“Functional sensitivity,” refers to the interassay precision obtained at very low analyte levels, for certain diagnostic assays with high precision requirements at low levels. “Analytical sensitivity,” is defined by the IUPAC as “the slope of the calibration curve.”

The term “linearity” refers to the ability to provide results that are directly proportional to the concentration (amount) of the analyte in the test sample. The range of linearity is the range of analyte concentration within which an assay exhibits linearity. The lower limit of linear range (LLR), is the lowest concentration at which the assay has a linear relationship with the true concentration.

The “dynamic range” of quantification of an assay refers to the range of analyte levels within which quantification of the analyte is accurate.

The terms Cₜ or Cₚ, as used interchangeably herein, are well known in the art to refer to the point at which a fluorescent or other signal in a real time PCR assay crosses a specified threshold, and nominally provide an estimate of the number of PCR cycles needed to amplify a nucleic acid analyte to a particular concentration.

In certain embodiments, analytes in a test sample can include substances useful for the detection of TNF alpha, HIV p24, and PSA.

As used herein, tumor necrosis factor (TNF, cachexin or cachectin, also known as tumor necrosis factor-alpha or TNF-α) is a cytokine involved in systemic inflammation and is a member of a group of cytokines that stimulate the acute phase reaction. It is produced chiefly by activated macrophages, although it can be produced by other cell types as well. TNF is involved in the regulation of immune cells. TNF is able to induce fever, to induce apoptotic cell death, to induce sepsis (through IL1 & IL6 production), to induce cachexia, induce inflammation, and to inhibit tumorigenesis and viral replication. Dysregulation of TNF production has been implicated in a variety of human diseases, including rheumatoid arthritis, Alzheimer's disease, cancer, major depression, and inflammatory bowel disease (IBD).

In certain embodiments, the p24 antigen test detects the presence of the p24 protein of HIV (also known as CA), the capsid protein of the virus. Presence of the p24 protein in the person's blood can be used to detect the presence of the virus in a subject.

In certain embodiments, the prostate specific antigen PSA test detects the presence of the Prostate-specific antigen (PSA), which is a member of the kallikrein-related peptidase family secreted by the epithelial cells of the prostate gland. Prostate-specific antigen (PSA) is a 28.4-Kd single-chain glycoprotein that belongs to the kallikrein family of serine proteases. In the prostate gland, it is produced by the epithelial lining of the acini and ducts. PSA measurements in serum, where the main immunoreactive forms occur as free PSA and as a complex with the proteinase inhibitor α1-antichymotrypsin (PSA-ACT), are widely used in the detection, diagnosis, and monitoring of patients with prostate cancer (Rafferty B, 200021).

PSA is present in small quantities in the serum of men with healthy prostates, but is often elevated in the presence of prostate cancer and in other prostate disorders. U.S. Application No. 20090246781, incorporated herein by reference in its entirety, describes compositions and methods for use in PSA assays, including immuno-PCR assays, which have a low functional sensitivity.

The following are examples of analytes that may be detected using the compositions, methods and kits of the present invention:

1. protamines

2. histones

3. albumins

4. globulins

5. scleroproteins

6. phosphoproteins

7. mucoproteins

The following examples are clinically important proteins found in human plasma that may be detected using the compositions, methods and kits of the present invention:

1. α1-Lipoprotein

2. α1-Antitrypsin

3. Transcortin

4. 4.6S-Postalbumin

5. Tryptophan-poor

6. α1-Glycoprotein

7. α1_{χ}-Glycoprotein

8. Thyroxin-binding globulin

9. Inter-α-trypsin-inhibitor

10. Gc-globulin

11. (Gc 1-1)

12. (Gc 2-1)

13. (Gc 2-2)

14. Haptoglobin

15. (Hp 1-1)

16. (Hp 2-1)

17. (Hp 2-2)

18. Ceruloplasmin

19. Cholinesterase

20. α2-Lipoprotein(s)

21. Myoglobin

22. C-Reactive Protein

23. α2-Macroglobulin

24. α2-HS-glycoprotein

25. Zn-α2-glycoprotein

26. α2-Neuramino-glycoprotein

27. Erythropoietin

28. β-lipoprotein

29. Transferrin

30. Hemopexin

31. Fibrinogen

32. Plasminogen

33. γ2-glycoprotein I

34. γ2-glycoprotein II

35. Immunoglobulin G

36. (IgG) or γG-globulin

37. Mol. formula: γ2k2 or β2λ2

38. Immunoglobulin A (IgA) or γA-globulin

39. Mol. formula: (α2κ2)n or (α2κ2)n

40. Immunoglobulin M (IgM) or γM-globulin

41. Mol. formula: (μ2κ2)5 or (μ2λ2)5

42. Immunoglobulin D (IgD) or γD-Globulin (γD)

43. Mol. formula: (.delta.2κ2) or (.delta.2λ2)

44. Immunoglobulin E (IgE) or γE-Globulin (γE)

45. Mol. formula: (ε2κ2) or (ε2λ2)

46. Free κ and λ light chains

Complement Factors:

1. C′1

2. C′1q

3. C′1r

4. C′1s

5. C′2

6. C′3

7. β1A

8. α2D

9. C′4

10. C′5

11. C′6

12. C′7

13. C′8

14. C′9

Important blood factors that may be detected using the compositions, methods and kits of the present invention include the examples listed below.

### Blood Factors

1. International

2. Designation Name

3. I Fibrinogen

4. II Prothrombin

5. IIa Thrombin

6. III Tissue thromboplastin

7. V and VI Proaccelerin, accelerator globulin

8. VII Proconvertin

9. VIII Antihemophilic globulin (AHG)

10. IX Christmas factor plasma thromboplastin component (PTC)

11. X Stuart-Prower factor, autoprothrombin III

12. XI Plasma thromboplastin

13. XIII Fibrin-stabilizing factor

14. CK-18

Important protein hormones that may be detected using the compositions, methods and kits of the present invention include:

1. Peptide and Protein Hormones

2. Parathyroid hormone (parathromone)

3. Thyrocalcitonin

4. Insulin

5. Glucagon

6. Relaxin

7. Erythropoietin

8. Melanotropin (melanocyte-stimulating hormone; intermedin)

9. Somatotropin (growth hormone)

10. Corticotropin (adrenocorticotropic hormone)

11. Thyrotropin

12. Follicle-stimulating hormone

13. Luteinizing hormone (interstitial cell-stimulating hormone)

14. Luteomammotropic hormone (luteotropin, prolactin

15. Gonadotropin (chorionic gonadotropin)

16. Tissue Hormones

17. Secretin

18. Gastrin

19. Angiotensin I and II

20. Bradykinin

21. Human placental lactogen

22. Cytokines

23. IL 1

24. IL 2

25. IL 4

26. IL 6

27. IL 8

28. IL10

29. EGF

30. TNF

31. NGF

32. Cancer Antigens

33. PSA

34. CEA

35. α-fetoprotein

36. Acid phosphatase

37. CA19.9

38. CA125

39. Tissue Specific Antigens

40. alkaline phosphatase

41. myoglobin

42. CPK-MB

43. Troponin

44. BNP

45. Pro-BNP

46. Calcitonin

47. Myelin basic protein

48. Peptide Hormones from the Neurohypophysis

49. Oxytocin

50. Vasopressin

51. Releasing factors (RF) CRF, LRF, TRF, Somatotropin-RF, GRF, FSH-RF, PIF, MIF

52. Ricin

53. Diptheria toxin

54. Botulism toxin

55. Staphylococcus enterotoxin B

56. Drugs or drug metabolites

Bacteria and viruses are also analytes that may be detected using the compositions, methods and kits of the present invention.

### Assay Kinetics

The invention herein is based in part on the following observations. In most immunoassay formats, antibodies are used at concentrations much higher than 10 pM. For example, typical reporter antibody concentration in the representative literature have detection antibody concentration ranging from 133 pM to 10,000 pM. (e.g., Zhou et al., NAR (1993) 21:6038; Hendrickson et al., NAR (1995) 23:522; Joerger et al., Clin Chem (1995) 41:1371; Sims et al., Anal Biochem (2000) 281:230; Furuya et al., J Immunol Meth (2000)238:173; McKie et al., J Immunol Meth (2002) 270:135; Rissin et al., Nature Biotechnol (2010) 28:595; Rissin et al., Anal Chem (2011) 83:2279; Song et al., J Immunol Meth (2011) 372:177). The molar concentration of reporter antibody present in the assays reported in the references was calculated, in some instances, based on the assumption that the reporter antibodies used in these studies were IgG antibodies, with molecular weights of approximately 150 Kd.

The hundred or thousand-fold excess antibody concentrations employed in these assays would be expected to drive the binding equilibrium between the reporter antibody and the analyte to a high fraction of analyte bound to the reporter antibody, resulting in increased molar concentrations of the reporter MAb-analyte complex, and an increased signal and higher sensitivity. Therefore, assays previously reported by others utilized a high reporter antibody concentration (for example, a 100-fold or 1000-fold greater reporter antibody concentration than the concentration disclosed herein) to drive the equilibrium of bound antigen to a very high fraction of the antigen population so that the molar amount of the antibody-antigen complex could be more readily detected.

The instant disclosure is based on the surprising observation that greater sensitivity is obtained in a two-site immunoassay using low concentrations of reporter antibodies (less than about 30 pM, preferably from about 3 pM to about 10 pM) in a forward assay orientation, using a highly sensitive label such as a nucleic acid label. The nucleic acid label and other highly sensitive labels are unique in that as low as single molecules of captured labeled-reporter can, in principle, be detected. The PCR label is also unique in that the marker DNA label is amplified during the detection step.

Although the use of low quantities of binding agent has been suggested for use in an array format assay, see Ekins, U.S. Pat. No. 5,432,099 and U.S. Pat. No. 5,599,720, the inventors of the invention disclosed herein believe that they are the first to demonstrate that concentrations of antibodies ranging from between about 0.1 pM to about 15 pM, between about 0.1 pM to about 10 pM, between about 3.0 pM to about 10 pM, between about 3.0 pM to about 15 pM, or between about 3.0 pM to about 30 pM (or other ranges of concentrations or concentrations of reporter antibody disclosed elsewhere herein) surprisingly yield a higher sensitivity in a forward immunoassay than the sensitivity obtained when using a reporter antibody at a higher concentration such as 100 pM. In addition, the Ekins disclosure focuses on assays for use with solid phase arrays, wherein the antigen is present in excess of the antibody—in direct contrast to the methodologies disclosed herein.

In the immunoassay arts, selection of antibodies based on equilibrium binding or dissociation constants. For example, the equilibrium dissociation constant, K_{d}, can be defined as follows:

$${K}_{d} = \frac{[ Free Antibody ] [ Free Antigen ]}{[ Antibody \cdot Antigen ]}$$

The units of K_{d} are Moles/Liter, or “M.”

The equilibrium association constant, Kₐ, which is well known in the art to be the reciprocal of K_{d} and contains the same information. Units for Kₐ are expressed as Liters/Mole or “M⁻¹.”

The smaller the K_{d}, the lower the concentration of free reactants needed to drive the equilibrium to a given concentration of the bound complex. For monoclonal antibodies, especially those useful for immunoassays, the measured K_{d} values for commonly used high affinity antibodies fall below 10⁻⁸, usually in the range of between about 10⁻⁸ M to about 10⁻¹⁰ M. Antibodies at the low end of this range (10⁻¹⁰), or lower, are conventionally regarded as having higher “affinity” and being more useful in immunoassays.

For quantitative assays involving binary interactions like antibody antigen complexation, it is common to have one of the reactants present in at least 10-fold excess relative to the other so that the relative concentration of the more abundant reactant essentially doesn't change as the reaction proceeds and the ultimate signal derived from the complexed analyte is approximately a linear function of the input analyte dose. This also simplifies the calculation of solving for the equilibrium concentration of the various species given known input amounts and equilibrium constants.

In this discussion, the following terms have the following definitions:

Ab=Ab concentration

Ag=Initial antigen concentration

B=Ab·Ag complex concentration

Ag—B=present free antigen concentration

K_{d}=Equilibrium dissoc constant

$$\frac{Ab ( Ag - B )}{B} = {K}_{d}$$

$$Ab \frac{Ag}{( Ab + {K}_{d} )} = B$$

Where B is the equilibrium concentration of the antigen antibody complex.

It is also well known in the art that in a chemical system like the antibody-antigen binding, equilibrium is achieved when the rate of the forward reaction, in this case dissociation of the antigen antibody complex into its constituents, is exactly the same as the rate of the reverse reaction, or association to form a complex. It thus follows that

$${K}_{d} = \frac{k 2}{k 1}$$

where k₁ is the rate constant for the forward reaction (in units of M⁻¹·s⁻¹) and k₂ is the rate constant for dissociation of complexes (in units of s⁻¹). k₁ is also referred to herein as the association constant or kₐ. k₂ is also referred to herein as the dissociation constant or k_{d}.

Given knowledge of the underlying rate constants, it is possible to model in a simulated assay the concentration of antigen bound by antibody at any time (Bₜ) after the antibody and antigen are first contacted at a specified concentration of antibody. As long as the antigen is present at a concentration significantly less than the antibody (e.g., where antigen is present at a 10-fold lower concentration than the antibody), it is not necessary to know the antigen concentration to predict what fraction of the total antigen will be bound in complex with antibody as shown here (viz., Bₜ/Ag):

$$\frac{d B}{d time} = [ k 1 \cdot Ab \cdot ( Ag - B ) ] - k 2 \cdot B$$

$$k 1 \cdot Ab \cdot ( Ag - B ) - k 2 \cdot B$$

$$k 1 \cdot Ab \cdot Ag - k 1 \cdot Ab \cdot B - k 2 \cdot B$$

$$k 1 \cdot Ab \cdot Ag - k 1 \cdot Ab \cdot B - k 2 \cdot B$$

$$k 1 \cdot Ab \cdot Ag - B \cdot ( k 1 \cdot Ab + k 2 )$$

$${\int }_{0}^{{B}_{t}} \frac{1}{[ k 1 \cdot Ab \cdot Ag - B \cdot ( k 1 \cdot Ab + k 2 ) ]} d B = {\int }_{0}^{t} 1 d time$$

$$Where Bt = amount of Antigen Bound at a given time = t$$

$$\frac{- ln ( - {B}_{t} \cdot k 1 \cdot Ab - {B}_{t} \cdot k 2 + k 1 \cdot Ab \cdot Ag )}{( k 1 \cdot Ab + k 2 )} + \frac{( ln ( k 1 ) + ln ( Ab ) + ln ( Ag ) )}{( k 1 \cdot Ab + k 2 )} = t$$

$$k 1 \cdot Ab \cdot Ag \cdot \frac{( exp ( t \cdot k 1 \cdot Ab + t \cdot k 2 ) - 1 )}{exp ( t \cdot k 1 \cdot Ab + t \cdot k 2 ) \cdot ( k 1 \cdot Ab + k 2 )} = {B}_{t}$$

$$Ag \cdot \frac{k 1 \cdot Ab}{( k 1 \cdot Ab + k 2 )} \cdot \frac{( exp ( t \cdot k 1 \cdot Ab + t \cdot k 2 ) - 1 )}{exp ( t \cdot k 1 \cdot Ab + t \cdot k 2 )} = {B}_{t}$$

$$Ag \cdot \frac{k 1 \cdot Ab}{( k 1 \cdot Ab + k 2 )} \cdot \frac{( exp ( t \cdot k 1 \cdot Ab + t \cdot k 2 ) - 1 )}{exp [ t \cdot ( k 1 \cdot Ab + k 2 ) ]} = {B}_{t}$$

$$Ag \cdot \frac{k 1 \cdot Ab}{( k 1 \cdot Ab + k 2 )} \cdot \frac{[ exp [ t \cdot ( k 1 \cdot Ab + k 2 ) ] - 1 ]}{exp [ t \cdot ( k 1 \cdot Ab + k 2 ) ]} = {B}_{t}$$

$$Ag \cdot \frac{k 1 \cdot Ab}{( k 1 \cdot Ab + k 2 )} \cdot \frac{[ exp [ t \cdot ( k 1 \cdot Ab + k 2 ) ] - 1 ]}{exp [ t \cdot ( k 1 \cdot Ab + k 2 ) ]} = {B}_{t} \\ Ag \cdot \frac{k 1 \cdot Ab}{( k 1 \cdot Ab + k 2 )} \cdot [ 1 - exp [ - t \cdot ( k 1 \cdot Ab + k 2 ) ] ] = {B}_{t} \\ \frac{k 1 \cdot Ab}{k 1 \cdot Ab + k 2} \cdot [ 1 - exp [ - t \cdot ( k 1 \cdot Ab + k 2 ) ] ] = \frac{{B}_{t}}{Ag}$$

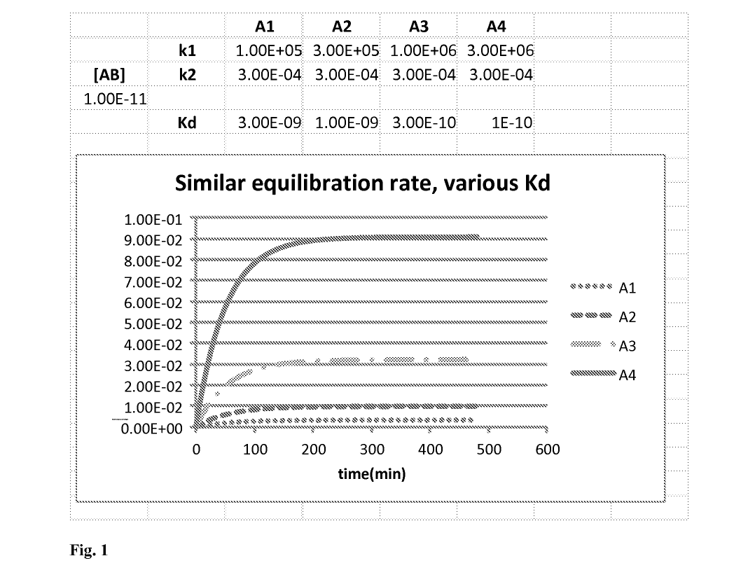

Because the K_{d} of an antibody determines the fraction of bound to free antigen at equilibrium, as discussed above, it is common in the immunoassay field to look principally at K_{d} of a monoclonal antibody to determine its suitability for use in an immunoassay. FIG. 1 is a graph of the fraction of total antigen bound over time as four simulated antibody—analyte (or antigen) reactions proceed to equilibrium. The reporter antibody present at a concentration of 10 pM in each simulated assay has a different K_{d} values, as shown in Table 1, from 3×10⁻⁹ to 1×10⁻¹⁰.

TABLE 1

| | A1 | A2 | A3 | A4 |
|---|---|---|---|---|
| k₁ | 1.00 × 10⁵ | 3.00 × 10⁵ | 1.00 × 10⁶ | 3.00 × 10⁶ |
| k₂ | 3.00 × 10⁻⁴ | 3.00 × 10⁻⁴ | 3.00 × 10⁻⁴ | 3.00 × 10⁻⁴ |
| K_{d} | 3.00 × 10⁻⁹ | 1.00 × 10⁻⁹ | 3.00 × 10⁻¹⁰ | 1.00 × 10⁻¹⁰ |

In this scenario, the rate at which equilibrium is achieved is approximately the same for each of the 4 antibodies, but the maximum fraction of antigen bound is different in all 4 cases (see FIG. 1 for bound antigen levels in the graph), ranging from about 0.0033 to about 0.091 over the K_{d} range above, respectively.

In conventional immunoassay practice the smallest K_{d} is frequently used as the basis for selecting a monoclonal antibody useful in an immunoassay because the fraction of bound antigen is expected to be the greatest. However, even for a high affinity antibody with a low K_{d}, e.g., 10⁻¹⁰, not all antibodies with that equilibrium constant are equivalent, since the K_{d} could be the quotient of a very fast dissociation rate and a very fast association rate, or alternatively, the quotient of a very slow dissociation rate and a very slow association rate, two situations that the inventors have determined yield very different results in forward immunoassays.

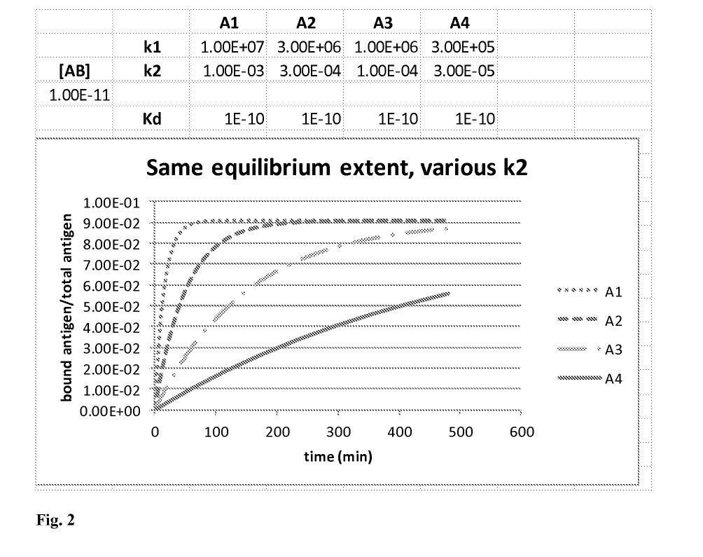

FIG. 2 is a graph of the fraction of total antigen bound over time in simulated binding reactions for four different antibodies at 10 pM having the same equilibrium binding constants (K_{d} of 10⁻¹⁰), but different association (kₐ) and dissociation constants (k_{d}), as shown in Table 2.

TABLE 2

| | A1 | A2 | A3 | A4 |
|---|---|---|---|---|
| k₁ | 1.00 × 10⁷ | 3.00 × 10⁶ | 1.00 × 10⁶ | 3.00 × 10⁵ |
| k₂ | 1.00 × 10⁻³ | 3.00 × 10⁻⁴ | 1.00 × 10⁻⁴ | 3.00 × 10⁻⁵ |
| K_{d} | 1.00 × 10⁻¹⁰ | 1.00 × 10⁻¹⁰ | 1.00 × 10⁻¹⁰ | 1.00 × 10⁻¹⁰ |

FIG. 2 illustrates that even for high affinity antibodies having a K_{d} of 10⁻¹⁰, not all of the binding reactions are expected to reach equilibrium by 500 minutes. Because each K_{d} depends on the association and dissociation constants, the times for the binding reactions to reach equilibrium can differ for antibodies having the same equilibrium binding constants, K_{d}. For this hypothetical set of 4 antibodies with the same K_{d}=10⁻¹⁰, all of the binding reactions will eventually reach the same equilibrium, i.e., the same ratio of bound antigen to total antigen, yielding a fraction of antigen bound=0.091. However for antibody A1 having a pair of rate constants (k1=1×10⁷ M⁻¹ s⁻¹, k2=10⁻³ s⁻¹) the maximum binding extent is achieved in about 1 hour whereas it will take more than 50 hours for antibody A4 with the slowest rate constants (k1=3×10⁵ M⁻¹ s⁻¹, k2=3×10⁻⁵ s⁻¹) to reach equilibrium. Some assay formats depend on achieving equilibrium to ensure a reproducible and stable signal response to dose. Many assay formats are configured to reach equilibrium, based on the assumption that the maximum possible signal will provide maximum sensitivity. Accordingly, in conventional assay design, selection of an antibody with a slow rate of equilibration (as in certain methods of this invention) over an antibody with a considerably more rapid rate of system equilibration is not obvious to one skilled in the art of immunoassay since, although small k_{d} values yield slow dissociation rates desirable for stable complexes, they also correspond to slow rates of equilibration, especially in conjunction with low antibody concentration. Nevertheless, as discussed further herein, it has been discovered that selection of an antibody with a slow rate of dissociation is especially advantageous for use with a forward two-site immunoassay using a nucleic acid label even though this means equilibration will occur more slowly and that state may never be achieved in a practical length of time.

### EXAMPLES

While various embodiments of the present invention have been described above, it should be understood that they have been presented by way of example only, and not limitation. The following non-limiting examples are illustrative of the invention.

### Example 1

### Assays

An embodiment of an assay described herein is a two site monoclonal antibody immunoassay for the quantitative measurement of analyte, including any of the analytes set forth above. In some embodiments the immunoassay will be a PCR immunoassay. The method is capable of precise and accurate detection of very low analyte concentrations in samples and can detect analyte present at concentrations present in samples at about 100 fg/mL and as low as about 10 fg/mL.

One exemplary immuno PCR assay of the instant invention employs a soluble (reporter) monoclonal antibody (MAb), labeled with an assay specific double-stranded DNA sequence. The presence of this DNA label does not interfere with MAb binding, and the MAb does not interfere with DNA label detection. The second capture MAb specific for another site on the analyte was coated onto paramagnetic microparticles.

The reporter MAb-DNA conjugate is reacted with sample in a microtiter plate format to form a first immune complex between the reporter MAb-DNA and the analyte. The first immune complex is then captured onto paramagnetic microparticles coated with the second capture MAb, forming an insoluble two-site immune complex. The microparticles are washed by several cycles of magnetic capture and re-suspension to remove excess reporter MAb-DNA conjugate, see FIG. 1.

The specifically bound DNA label is then detected by subjecting suspended particles to PCR conditions and monitoring the generations of amplicon in real time. Quantitation of the analyte is performed by monitoring the number of PCR thermocycles it takes to generate a predetermined fluorescent signal over baseline. This is accomplished by automatic instrumentation designed to monitor fluorescence intensity as a function of cycle number. The amount of DNA initially present in the sample is inversely proportional to the threshold cycle (Ct). Concentration of analyte (pg/mL or fg/mL) in each sample is then calculated from the calibration curve.

### Immuno-PCR Assay Procedure

A 20 μL sample is placed in a well of a sample holder such as a 96-well microtiter plate, followed by addition of 75 μL of reporter specificity molecule, for example, MAb-DNA, to final desired concentration of reporter specificity molecule. For example, a set of assays may be performed using 1, 3, 5 or 10 pM reporter Mab or other reporter specificity molecule. The 96-well microtiter plate may be an AB 96-well Microamp plate. Calibrator and control samples may also be included in the assay run.

After addition of the reporter specificity molecule, the sample holder is securely covered, placed on a platform shaker and gently agitated at approximately 500 rpm for one minute to thoroughly mix reactants. The reaction is allowed to proceed for an additional two hours at room temperature without mixing. The plate is uncovered and 10 μL (5 ug) of a suspension of paramagnetic micro particles coated with second capturing antibody is added. The 96 well plate is covered with another seal and placed on a platform shaker at 500 rpm and incubated for 30 minutes at room temperature.

The sample holder, e.g., the AB 96-well plate, is placed onto a magnetic rack and four sequential wash and separation steps are performed using a 125 μL wash solution composed of a Tris buffered solution of normal saline. The plate is centrifuged at 450-500×g for one minute after the first and third wash and separation steps. A wash procedure using an automated micro plate washer is also suitable.

### PCR Reagent Addition Step

30 μL of PCR Reagent is added to each well. The sample holder is covered. For example, a plate or other holder may be covered with an optical adhesive cover then centrifuged at 450-500×g for one minute at a minimal brake setting.

Detection is accomplished by performing 30 to 40 cycles of qPCR in an appropriate real time PCR device, such as the AB 7500 Fast Dx or any other device known in the art.

The concentration of analyte is determined by comparison and extrapolation from appropriate control samples using the threshold cycle. The PCR results may be analyzed using a manual count setting and auto baseline setting, or may also be analyzed using time or an algorithm to create a calibration curve and calculate the analyte concentration.

### Example 2

### HIV p24 Assay

It was surprisingly found in the assays of this invention that using a reporter antibody having a relatively slow dissociation rate constant gave rise to an exquisitely sensitive assay. For example, it was surprisingly discovered that a two-site immuno-PCR assay using an antibody with a dissociation rate constant less than 5.9×10⁻⁵ sec⁻¹ provided an assay capable of detecting at least as low as 0.1 fM of analyte—orders of magnitude lower than other reported assays.

It was thus surprisingly discovered that the rate of equilibration of analyte binding may be determined primarily by k₂, rather than k₁ or K_{d} and/or any combination of reactant concentrations, and that the population of unbound analyte approximates a first order exponential decay from 100% unbound analyte to the equilibrium fraction of bound analyte. It is also surprisingly found that a relatively slow dissociation rate constant is critical to assay design and may be more critical to assay sensitivity than the equilibrium constant (K_{d}), even when comparing an antibody with the lowest obtained dissociation rate constant to an antibody having a higher affinity/lower K_{d}. The advantages of using an antibody with the slowest dissociation constant are especially realized when performing immunoassays using the low antibody concentrations preferred in the methods of this invention.

### HIV p24 Assay

A two site monoclonal antibody immuno-PCR assay for the quantitative measurement of HIV p24 in human serum was performed. The method was capable of precise and accurate detection of very low p24 concentrations in biological samples such as serum and detected p24 present at concentrations present in samples at about 100 fg/mL and as low as about 1 fg/mL.

The assay used a soluble (reporter) monoclonal antibody (MAb) p24, labeled with an assay specific double-stranded DNA sequence. The capture MAb specific for another site on p24 was coated onto paramagnetic microparticles.

An oligonucleotide having the following sequence was used to form the DNA-reporter antibody conjugate:

The reporter antibody was NIH AIDS Research & Reference Reagent Program HIV-1 183-H12-5C with a unique single strand DNA attached.

Reporter Sequence:
(SEQ ID NO: 1)
5′-{C12 NH2}TGCGTAGCGATGACTAGCTGCTGATCGATATTAGCTAG
CATCAGCGATCGATACGAGCA-3′

An oligonucleotide having the following sequence is complementary to the reporter sequence, and hybridizes with the reporter antibody-reporter sequence conjugate to form a double stranded hybrid attached to the reporter antibody.

Reporter Sequence Complement:
(SEQ ID NO: 2)
5′-TGCTCGTATCGATCGCTGATGCTAGCTAATATCGATCAGCAGCTAGT
CATCGCTACGCA-3′

The reporter DNA can be amplified by PCR using the following primers, or any other suitable primers:

Primer 1 Sequence:
(SEQ ID NO: 3)
5′-TGCGTAGCGATGACTAGCTGCTG-3′
Primer 2 Sequence:
(SEQ ID NO: 4)
5′-TGCTCGTATCGATCGCTGATGCT

Capture antibody was made using Maine Biotechnology MAb 739P, which was biotinylated and attached to streptavidin magnetic particles. For example, the HIV p-24 capture system was prepared by labeling a monoclonal antibody to p24, designated as 739p, from Maine Biotechnology Services with sulfo-NHS-PEG (3400 MW)-biotin from Nanocs (NY, NY) to a level of 5 biotin/mole. Oligonucleotides of 60 bases were synthesized to contain a functional amine attached to the 5′ end through a 12-carbon spacer arm from Glen Research Corp. (Sterling, Va.) and purified by preparative polyacrylamide gel electrophoresis. The 5′ amino function was activated with a 100-fold excess of disuccinimidyl suberate to minimize cross-linking. The intermediate was rapidly purified by gel-filtration fast protein liquid chromatography (FPLC) in 5 mmol sodium citrate (pH 5.4) in order to maintain the second succimidyl function. The DNA was concentrated by centrifugal ultrafiltration at 4° C. and combined immediately at room temperature with 10 mg/ml antibody in 0.3 mol phosphate buffer (pH 8) and 0.45 mol NaCl for 1 hour. Unreacted antibody was removed by size-exclusion FPLC using a Superose S-200 column from GE Healthcare (Piscataway, N.J.) that had been equilibrated in Tris-buffered saline (pH 7.4). Unreacted oligonucleotide was removed by anion-exchange FPLC using a Mono Q column from GE Healthcare and 5%/min salt-gradient elution to 1 mol in 20 mmol Tris (pH 7.4). Typically, 50% of the protein was recovered as conjugate.

Both native and sodium dodecyl sulfate (SDS) gel electrophoresis revealed the presence of antibody containing predominantly one or two strands of the 60-mer. The presence of covalent antibody does not interfere with PCR signal. Likewise, the DNA label does not obstruct binding to antigen, as determined by HRP-labeled second-antibody detection of solid-phase antigen.

Recombinant HIV-1 p24 was prepared by diluting into BSA/PBS.

The reporter antibody diluent was Modified Dulbecco Phosphate buffered saline pH 7.4 containing:
150 mM Sodium Chloride
1 mM EDTA
1% Poloxamer 338
0.09% Sodium Azide

The wash solution was 10 mM Tris pH8.0 containing:
0.05% Tween 20
150 mM sodium Chloride
0.09% Sodium Azide

The PCR reagent was Roche 1×PCR reagent.

The reporter MAb-DNA conjugate was contacted with sample in a microtiter plate format and incubated for 120 minutes to allow formation of a first immune complex with p24. The immune complex was then captured onto paramagnetic microparticles coated with the second capture MAb, forming an insoluble sandwich immune complex. Incubation of the first immune complex with the capture MAb took place for 30 minutes. The microparticles were then washed by several cycles of magnetic capture and re-suspension to remove excess reporter MAb-DNA conjugate.

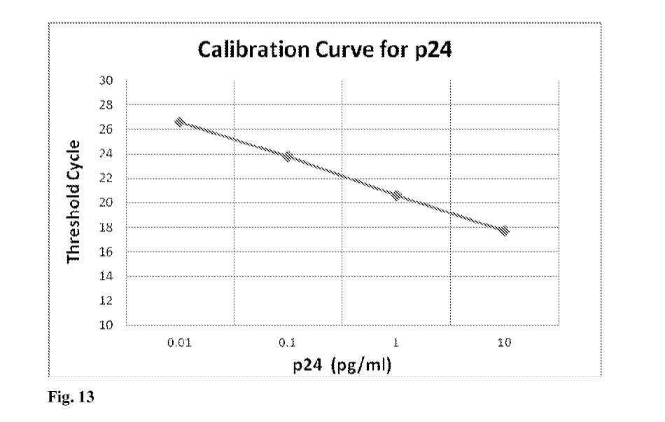

The DNA label was then detected by subjecting suspended particles to PCR conditions and monitoring the generations of amplicon in real time. Quantitation of p24 was performed by monitoring the number of PCR thermocycles required to generate a predetermined fluorescent signal over baseline. This was accomplished by automatic instrumentation designed to monitor fluorescence intensity as a function of cycle number. The amount of DNA initially present in the sample is inversely proportional to the threshold cycle (Ct). p24 concentrations (pg/mL or fg/mL) of the samples are calculated from the calibration curve (See FIG. 13).

A summary of the data for the calibration curve is shown below in Table 3.

TABLE 3

| pg/mL of p24 | Threshhold Cycle (Ct) |
|---|---|
| 0.01 | 26.6 |
| 0.1 | 23.8 |
| 1 | 20.6 |
| 10 | 17.7 |

### Assay Procedure

A 20 μL sample was placed in a well of a 96-well microtiter plate, followed by 75 μL of MAb-DNA to final concentration of 10 pM. The plate was securely covered, placed on a platform shaker and gently agitated at approximately 500 rpm for one minute to thoroughly mix reactants. The reaction was allowed to proceed for an additional two hours at room temperature without mixing. The plate was uncovered and 100 μL (5 ug) of a suspension of paramagnetic micro particles coated with second capturing antibody is added. The 96 well plate was covered with another seal and placed on a platform shaker at 500 rpm and incubated for 30 minutes at room temperature.

The AB 96-well plate was placed onto a magnetic rack and four sequential wash and separation steps were performed using a 125 μL wash solution composed of a Tris buffered solution of normal saline with 0.05% sodium and 0.5% Tween-20, pH 7.4. The plate was centrifuged at 450-500×g for one minute. A wash procedure using an automated micro plate washer is also suitable.

### PCR Reagent Addition Step

30 μL of PCR Reagent was added to each well. The plate was covered with an optical adhesive cover then centrifuged at 450-500×g for one minute at a minimal brake setting.

Detection was accomplished by qPCR in the AB 7500 Fast Dx.

The thermocycling profile was as follows:

1 1 cycle at 95.0° C. for 0:30 seconds
2 35 cycles at 62.0° C. extension for 0:30 seconds, followed by 95.0° C. for 15 seconds

The concentration of analyte is determined by comparison and extrapolation from appropriate control samples using the threshold cycle.

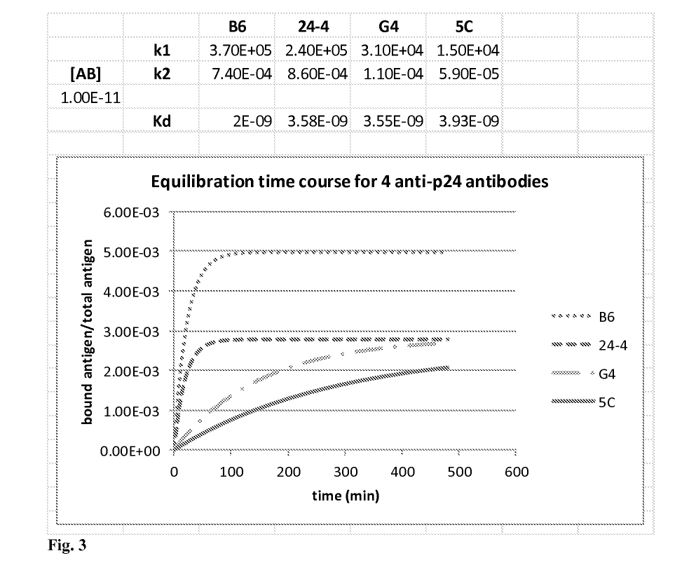

FIG. 3 is a graph the fraction of total antigen bound over time as four simulated antibody+analyte reactions proceed to equilibrium for 4 different commercially available antibodies specific for HIV p24 protein. The rate and equilibrium constants for the four antibodies are described in Table 4. The concentration of each antibody was 10 pM.

TABLE 4

| | B6 | 24-4 | G4 | 5C |
|---|---|---|---|---|
| k₁ | 3.70 × 10⁵ | 2.40 × 10⁵ | 3.10 × 10⁴ | 1.50 × 10⁴ |
| k₂ | 7.40 × 10⁻⁴ | 8.60 × 10⁻⁴ | 1.10 × 10⁻⁴ | 5.90 × 10⁻⁵ |
| K_{d} | 2.00 × 10⁻⁹ | 3.58 × 10⁻⁹ | 3.55 × 10⁻⁹ | 3.93 × 10⁻⁹ |

As shown, the K_{d} values for three antibodies (24-4, G4 and 5C) are very similar and the K_{d} for B6 is about 2-fold smaller than the others. On the other hand the dissociation rate constants, k₂, vary over >10-fold range and exhibit a broad range of equilibration times, from about 100 min for the fastest antibody to tens of hours for the slowest. As shown by the time course model of antigen (p24) binding in FIG. 3, the B6 antibody yields the highest levels of p24 binding at any given time point. Furthermore, its k₂ value is nearly equal to the maximum of the k₂ constants for the four antibodies.

Thus based solely on achieving the fastest, maximum equilibrium binding of p24, B6 might appear to be the most preferred antibody to use as a reporter antibody in a conventional immunoassay.

However, when tested, antibody 5C provides extremely high sensitivity, permitting detection of p24 at levels as low as 1 fg/mL. In addition, empirical testing of the 4 antibodies in a forward-sandwich immunoassay employing a low, 10 pM, concentration of reporter antibody, surprisingly reveals that antibody 5C gives the best results for robust signal to noise even though antibody 5C had both the worst K_{d} (largest K_{d} value and lowest affinity, with the smallest predicted bound fraction of p24 antigen at equilibrium), as well as the smallest k₂, predicting the slowest rate of equilibration.

### Example 2A

### Determination of Optimal Concentration for the Reporter Antibody-DNA Conjugate for HIV p24 Detection by Immuno-PCR

This experiment demonstrated an exemplary assay for the detection of HIV p24. The experiment was designed to determine the signal differences between 1% BSA/PBS/0.1 mg/mL, Salmon Sperm blanks, and the same matrix spiked with 0.1 pg/mL HIV p24 when assayed using various concentrations of reporter antibody (reporter anti-HIV p24 Ab) between 2.5 and 50 pM.

### Methods:

50 uL of reporter Ab stock was prepared by diluting BSA/PBS.

25 uL of BSA/PBS, or 0.1 pg/mL HIV p24 in BSA/PBS was loaded into appropriate wells (triplicate determinations for each reporter Ab concentration tested including a zero reporter Ab condition).

75 uL of appropriate reporter Ab mix was added to each well, and the mixture was incubated for 2 hrs.

10 uL of Target Capture Reagent was added and incubated for 30 min with mixing on platform shaker at 500 rpm.

The particles were separated and washed extensively with Wash buffer.

PCR mix was added to each well, the plate was covered with sealing foil and centrifuged for 1 minute at 500×g, and RT PCR was performed.

Results: from HIV p24 Assay

TABLE 5
Average ΔThreshold Cycle (Cp)

| | ΔCp when HIV |
|---|---|
| Reporter [MAb] (pM) | p24 = 0.1 pg/mL |
| 2.5 | 0.3 |
| 5 | 2.6 |
| 10 | 2.2 |
| 15 | 2 |
| 20 | 1.5 |
| 50 | 0.9 |

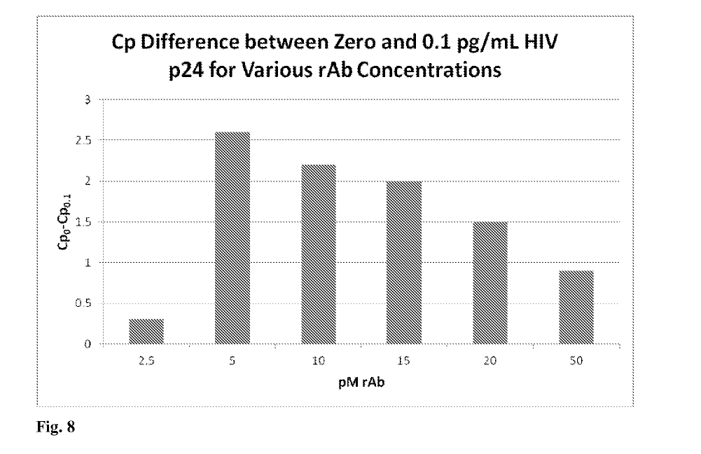

FIG. 8 is a graph describing the signal obtained above background for the HIV p24 results described in Table 5 above. In this example, the optimal concentration for the reporter antibody-DNA conjugate for HIV p24 detection is shown for the HIV p24 analyte at 0.1 pg/mL.

Surprisingly, the assays using reporter antibody concentrations of 5.0, 10.0 or 15.0 pM HIV p24 reporter antibody gave higher signal above background levels than the assay using 20 or 50 pM reporter antibody. In one embodiment, the optimal signal to noise is obtained with an input concentration of 10 pM MAb-DNA.

The antibody concentrations and kinetic properties taught as preferred in this disclosure are especially advantageous when used to design a forward sandwich immuno PCR assay, exemplified by the HIV p24 assay. Given the teaching of the instant disclosure, those of ordinary skill in the art will appreciate that the forward immunoassays of this invention, using, in some embodiments, a PCR label, an antibody having a low dissociation constant, and/or a low reporter antibody concentration, can be used to detect any analyte capable of being specifically bound by two specificity molecules.

In a forward immunoassay, an antigen or other detectable analyte is first contacted with a reporter antibody or reporter specificity molecule. The reporter antibody or specificity molecule is linked to a moiety that can be detected either directly or indirectly as evidence of the presence of that antibody. The analyte and antibody are incubated together for a length of time which is consistent with user needs and with sufficient assay performance.

During this first incubation period some of the reporter antibody or specificity molecules associate with some of the analyte molecules to form a reversible complex. During this time, the fraction of the analyte population bound by antibodies can be estimated using the kinetic modeling describe above. At the end of the specified incubation time, the mixture is further contacted with a second capture specificity molecule, such as an antibody, that is specific for a different target on the analyte molecule than is recognized by the first reporter specificity molecule. The capture specificity molecule, such as a capture antibody, may be linked to a solid support or other means of separating the two-site complex (comprising, for example, the two-site complex comprising the reporter specificity molecule or antibody, analyte and capture specificity molecule or antibody), from the rest of the solution that contains them, including the solute molecules and the rest of the molecules in solution such as unbound reporter specificity molecule or antibody.

The method must also provide a means of separating the solid phase-bound two-site complexes from the bulk phase of the solution, such as, for example, magnetic fields for paramagnetic particles, or filtration, gravity or centrifugation, reversible or irreversible chemical interactions with vessel walls, etc. Use of a magnetic field to concentrate, collect and reversibly immobilize paramagnetic particles is used in some embodiments in the instant application. When the solid phase comprising the capture specificity molecule is sufficiently sequestered from the bulk of the solution phase, the solution can be removed from contact with the particles, or alternatively the particles removed from the solution which contains other unbound molecules, such as non-specifically bound analyte and non-specifically bound reporter specificity molecules.

The bulk solution thus removed should contain most of the reporter specificity molecules not specifically bound into the two-site complex. After any one such separation, or wash, the fraction comprising the two-site complex will possibly still contain unbound reporter specificity molecules, and the vessel that contains this fraction may also have one or more components of the assay mixture, including reporter specificity molecules, non-specifically adsorbed to surfaces therein. Thus, in one embodiment, more than one cycle of these washing steps are performed, permitting high efficiency recovery of the two-site complexes during each wash cycle while the unbound or non-specifically bound components are further diluted and removed, especially reporter specificity not bound or not specifically bound to analyte, that incidentally copurify with the solid support during any one wash cycle.

After the first bulk solution removal, the majority of reporter specificity molecule not complexed with analyte (antigen) and not further complexed with the capture specificity molecule, is removed, together with the fraction of the analyte population that was not captured. This essentially eliminates the binding reaction (whose rate was described in the exemplary model above using antibody-antigen binding as=k1 [Free Specificity Molecule][Free Analyte], or =k1·Ab·(Ag−B).

Thus the only reaction that occurs once the first wash step is performed is the dissociation reaction, at a rate given by k₂·B, as in the models above.

Given an amount of complexed species Nₜ₁ at time=t1, the amount still complexed at a sunsequent time=t2 will be given by Nₜ₁·exp(−k2·(t2−t1)).

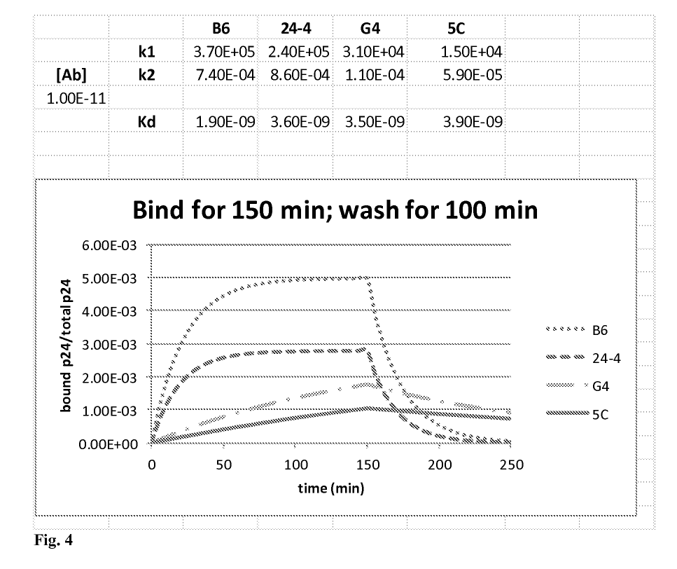

FIG. 4 is a graph describing the predicted fraction of bound p24 antigen for the four antibodies shown in FIG. 3 under simulated assay conditions where binding of the reporter antibody to antigen takes place during the 120 minute incubation period, with a 30 minute further incubation with the capture antibody after a small relative volume of capture antibody is added to the first reaction, and a series of multiple washes and incubations is performed to dilute and remove reporter antibody not bound in complex with both antigen and capture antibody. As shown, during the 120 minute incubation step, binding by antibody B6 reaches equilibrium. As indicated, although binding by antibody 5C never reaches equilibrium, and the maximum antigen binding by antibody 5C reaches only about ⅕ of the equilibrium level attained by antibody B6, markedly more antigen remains bound to antibody 5C after 50-100 minutes of washing than for antibody B6. Although antibodies 24-4, G4 and 5C all have similar K_{d} values, and thus similar predicted equilibrium extents, the extent of antigen binding attained by G4 and 5C never exceeds about ⅔ that of 24-4, or about ⅓ that of B6.

Nevertheless both G4 and 5C antibodies have properties that result in higher levels of bound analyte after even a fairly brief washing period following the binding incubations. As noted, the model illustrated in FIG. 4 assumes that the 120 min incubation with the reporter antibody is followed by a 30 min further incubation after a small relative volume of capture antibody is added to the first reaction, and then a series of washing steps. Although antibodies 24-4, G4 and 5C all have similar K_{d} values, and thus similar predicted equilibrium binding fractions of bound p24 to total p24, of those three antibodies, only binding by antibody 24-4 actually reaches equilibrium during the 120 minute incubation period. On the other hand the relatively low k_{d} of the 5C and G4 antibodies appears to allow a greater percentage of the complexes formed between the reporter antibody and antigen to remain intact during the wash step, than with the 24-4 and B6 antibodies. Thus, the empirically determined results can be explained with this model

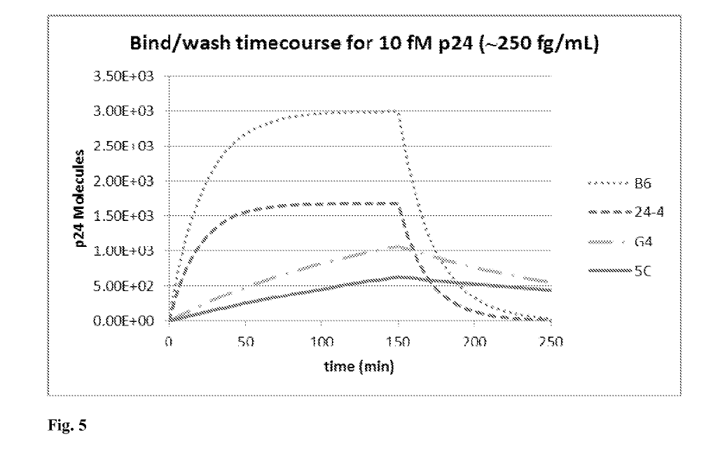

FIG. 5 is a graph describing the same assay time course estimated from the kinetic constants for the 4 HIV antibodies but the amount of p24 binding is converted to the estimated number of p24 molecules expected to be bound to antibody given an initial p24 concentration of 250 fg/mL in the sample being analyzed. As in FIG. 4, all of these calculations assume that the reporter antibody concentration is 10 pM=1×10⁻¹¹ M. Since the specificity molecule in these calculation is the reporter antibody, the number of p24 molecules shown is also an estimate of the number of reporter antibodies, thus in turn the number of labels, for example, oligonucleotide tags available to be detected, for example, following amplification by PCR. The number of labels available for detection drops to the order of hundreds. As explained by this model, antibodies G4 and 5C surprisingly retain a 10-50-fold stronger signal than antibody B6, despite the G4 and 5C antibodies binding reactions never reaching binding equilibrium, and G4 and 5C antibodies having lower affinity (higher K_{d}) properties. The maximum fold differences in signal between G4 and B6, and between 5C and B6 tracks the differences in dissociation constants of the antibodies. However, the slow dissociation constants for G4 and 5C help to stabilize the two-site complex during the washing steps necessary to reduce the amount of uncomplexed reporter specificity molecule in the system. Removing the non-specifically bound or unbound reporter specificity molecule is especially important when using a PCR label, since PCR will amplify any signal due to non-specific binding.

The models described herein are exemplary and are presented to illustrate the principles underlying the present invention. The models further help to highlight the surprising nature of the instant discovery that antibodies with slower dissociation rate constants provide greater sensitivity in non-equilibrium, two-site forward assays employing analyte binding followed by washing, even though the slow dissociation rate constants mean that equilibrium binding might not be reached during the assay.

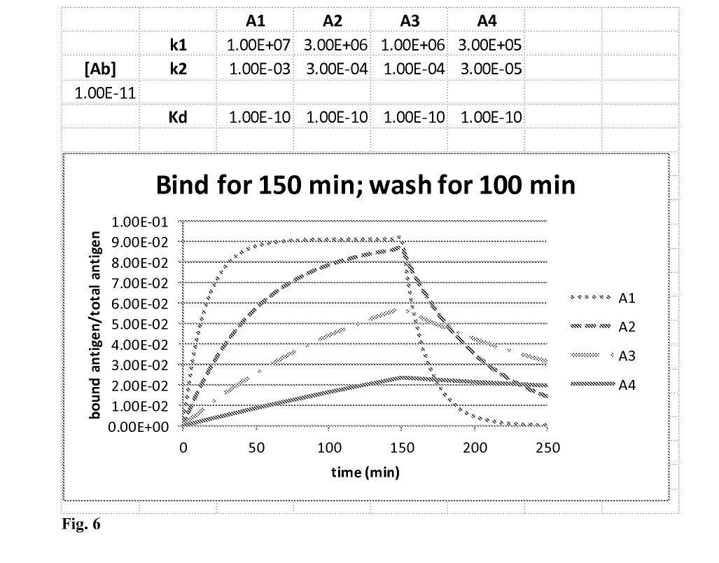

Monoclonal antibodies with equilibrium constants of K_{d}≦10⁻¹⁰ M have been reported, but such high affinity antibodies are not common without further antibody engineering. Because of the extremely small K_{d} such an antibody is regarded as desirable in conventional immunoassays. However FIG. 6 shows an example of how even such a very high affinity antibody might be a poor choice for a forward sandwich immunoassay, especially when employed as a reporter antibody at a concentration on the order of about 10 pM.

FIG. 6 is a graph describing simulated fraction of total antigen bound over time for high affinity monoclonal antibodies with equilibrium constants of K_{d}≦10⁻¹⁰ M but with different association (kₐ) and dissociation constants (k_{d}), as described in Table 6. This model assumes reporter antibody at a concentration on the order of about 10 pM.

TABLE 6

| | A1 | A2 | A3 | A4 |
|---|---|---|---|---|
| k₁ | 1.00 × 10⁷ | 3.00 × 10⁶ | 1.00 × 10⁶ | 3.00 × 10⁵ |
| k₂ | 1.00 × 10⁻³ | 3.00 × 10⁻⁴ | 1.00 × 10⁻⁴ | 3.00 × 10⁻⁵ |
| K_{d} | 1.00 × 10⁻¹⁰ | 1.00 × 10⁻¹⁰ | 1.00 × 10⁻¹⁰ | 1.00 × 10⁻¹⁰ |

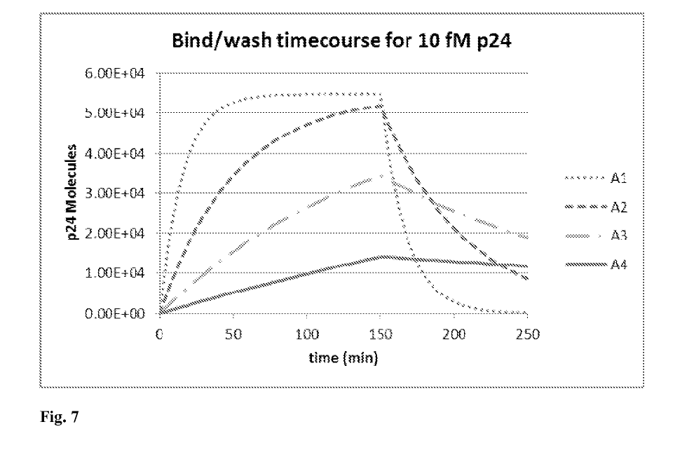

Because of the extremely small K_{d} each antibody might be regarded in conventional immunoassay practice as nearly ideal. However as shown in the figure, not every one of these antibodies is a suitable choice for a forward sandwich immunoassay, especially when employed as a reporter antibody at a concentration on the order of about 10 pM. The antibodies represented in FIG. 6 each have a K_{d}=10⁻¹⁰ M characteristic of a high affinity antibody, but the k₂ and k₁ values for each antibody cover a range of k₂ values (which in turn fixes k₁ to be=k₂/K_{d}). As indicated, antibody A1, an antibody which rapidly reaches maximum equilibrium antigen binding, can be highly unstable to even brief routine washing, and the amount of bound antigen drops off rapidly to a miniscule fraction of the fully bound level. This can be seen more easily when the bound fraction values are converted to molecules of antigen bound for a 10 fM p24 analyte concentration (=about 250 fg/mL of HIV p24), as shown in FIG. 7.

FIG. 7 is a graph describing the fraction of total antigen bound over time for antibodies having the same kinetic parameters of FIG. 6, if those antibodies were used in an assay for HIV p24. The bound fraction values are expressed in terms of molecules of antigen bound for a 10 fM (=about 250 fg/mL for HIV p24) input level of analyte. As indicated, the number of antigen molecules bound by reporter A1 antibody drops precipitously to some tens of molecules over this time course and is at a far lower level than all of the other antibodies when the wash time exceeds about 30 minutes.

All three of the other antibodies (A2-A4) give robust signals throughout the full 100 minute wash interval simulated in this kinetic model. Therefore, based on the disclosure herein, the antibodies with dissociation rate constants of 3×10⁻⁴ sec⁻¹ or lower are useful as reporter antibodies in the forward two-site immunoassay format.

Although the kinetic constants for antibodies A2, A3 and A4 in the models in FIGS. 6 and 7 all appear to yield the potential for robust signal, this does not imply that they are all equally preferable. Individual samples (e.g., in different tubes or wells) that one wishes to compare might in the normal course of processing be subject to slightly different factors affecting wash extent. A few minutes difference in wash exposure will be expected to have a more profound difference in the amount of bound antibody·antigen for an antibody with a faster dissociation rate than for an antibody with a slower dissociation.

Thus the antibodies for use in the assays of this invention, having slow dissociation constants, further surprisingly yield more robust, linear results providing a more accurate measure of analyte concentration even in assay runs with significant differences in washing times that might be occasioned by normal processing and handling.

Therefore, according to the instant disclosure, for the set of high affinity (small K_{d}) antibodies in Table 4, antibody A4 is a preferred antibody. Although A4's maximum binding extent is only about ¼ of that exhibited by A1 and A2, and about ⅓ that exhibited by A3, its signal following washing is still very strong and this signal should be the most robustly quantitative. Based on the considerations disclosed and discussed herein, it is preferable in one embodiment that the value of k₂ fall in the range 1×10⁻⁵ to 3×10⁻⁴ sec⁻¹. It is more preferable that k₂ fall in the range of 1×10⁻⁵ to 1×10⁻⁴ sec⁻¹. Other preferred values for the dissociation constant of the reporter antibody for use in the assays of this invention are discussed elsewhere herein.

### Example 3

### Assay for TNF Alpha

### Example 3A

### Determination of Optimal Concentration for the Reporter Antibody-DNA Conjugate for TNF Alpha Detection by Immuno-PCR

This experiment demonstrated an exemplary assay for the detection of TNF alpha. The experiment was designed to determine the signal differences between 1% BSA/PBS/0.1 mg/mL, and the same matrix spiked with 5 pg/mL and 10 pg/mL TNF when assayed using various concentrations of reporter antibody (reporter anti-TNF-alpha Ab) between 1 and 1000 pM.

### Materials:

1. 1% BSA/PBS

2. TNF reporter Ab

3. TNF antigen (200 ng/mL)

4. Anti-TNF magnetic particles

5. PCR Master Mix; M00742 (Iris International)

6. Wash Buffer

### Methods:

1 mL of 2 ng/mL TNF was prepared by diluting 10 uL 200 ng/mL TNF into 990 uL BSA/PBS.

1 mL of 10 pg/mL TNF was prepared by diluting 5 uL of 2 ng/mL TNF into 995 uL BSA/PBS.

1 mL of 5 pg/mL TNF was prepared by diluting 2.5 uL of 2 ng/mL TNF into 997.5 uL BSA/PBS.

50 uL of 1187 pM reporter Ab stock was prepared by diluting 5 uL of 11,866 pM reporter Ab into 45 uL BSA/PBS.

1.2 mL of reporter Ab was prepared by diluting appropriate volumes of either 11,866 pM rAb or 1187 pM rAb stock into 1% BSA/PBS as shown in Table 7 below:

TABLE 7

| Desired rAb | reporter anti- | | |
|---|---|---|---|
| concentration | TNF-alpha MAb | BSA/ | |
| (pM) | stock (uL) | PBS | |
| 1000 | 101 | 1099 | rAb stock = 11,866 pM rAb |
| 300 | 30 | 1170 | rAb stock = 11,866 pM rAb |
| 100 | 10.1 | 1190 | rAb stock = 11,866 pM rAb |
| 30 | 3.03 | 1197 | rAb stock = 11,866 pM rAb |
| 10 | 1.01 | 1199 | rAb stock = 11,866 pM rAb |
| 3 | 3.03 | 1197 | rAb stock = 1187 pM rAb |
| 1 | 1.01 | 1199 | rAb stock = 1187 pM rAb |

25 uL of BSA/PBS, 5 pg/mL, or 10 pg/mL TNF in BSA/PBS was loaded into appropriate wells (triplicate determinations at each TNF level for each reporter Ab concentration tested including a zero reporter Ab condition).

75 uL of appropriate reporter Ab mix was added to each well, and the mixture was incubated for 2 hrs.

10 uL of Target Capture Reagent was added and incubated for 30 min with mixing on platform shaker at 500 rpm.

The particles were separated and washed extensively with Wash buffer.

PCR mix was added to each well, the plate was covered with sealing foil and centrifuged for 1 minute at 500×g, and RT PCR was performed.

### Results: From TNF Alpha Assay

TABLE 8
Average of Threshold Cycle (Cp)

| | Cp for TNF Dose: | | |
|---|---|---|---|
| Reporter [MAb] (pM) | 0 pg/mL | 5 pg/mL | 10 pg/mL |
| 0 | 30.00 | 30.00 | 30.00 |
| 1 | 30.00 | 25.22 | 24.11 |
| 3 | 30.00 | 23.54 | 22.73 |
| 10 | 29.84 | 21.91 | 20.93 |
| 30 | 27.69 | 20.61 | 19.59 |
| 100 | 26.01 | 19.94 | 18.83 |
| 300 | 23.77 | 19.69 | 18.66 |
| 1000 | 20.53 | 18.52 | 18.28 |

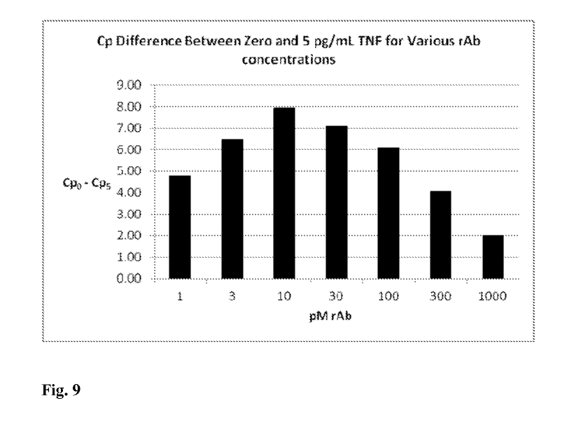

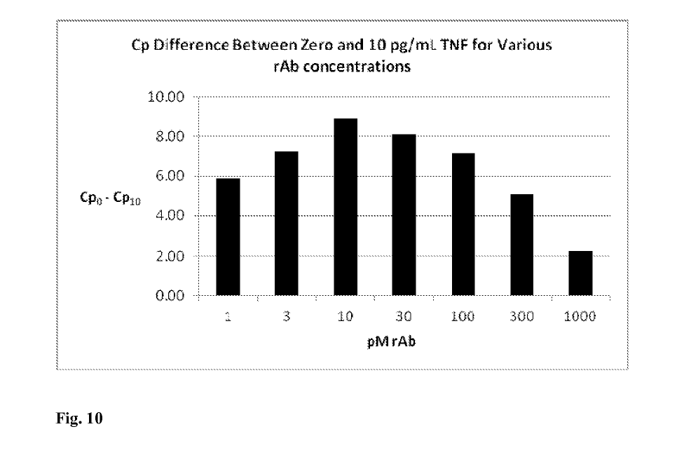

FIG. 9 is a graph describing the signal obtained above background for the TNF 5 pg/mL results described in Table 8 above. FIG. 10 is a graph describing the signal obtained above background for the TNF 10 pg/mL results described in Table 7 above. In this example, the optimal concentration for the reporter antibody-DNA conjugate for TNF alpha detection is shown for the TNF alpha analyte concentration up to 5 pg/mL. The Cp difference between zero TNF and 5 and 10 pg/mL TNF increases with decreasing rAb concentration up to about 10 pM.

Surprisingly, 3 pM reporter antibody gives a higher measured signal above background than 100 pM reporter antibody.

Multiple antibodies to TNF alpha are obtained, with K_{d} values of less than about 1×10⁻⁸ M. The K_{d}, kₐ, and k_{d} values for each antibody are obtained using any known method, such as using a Biacore system. Each of the antibodies is used as a reporter antibody in an immuno-PCR assay as described above. The concentration of each reporter antibody is the same in each set of assays, and that concentration may be 3, 10 or 30 pM.

### Example 4

### Exemplary Comparison of Reverse and Forward Assay Formats Using a Low Concentration Reporter Antibody for TNF-Alpha

To study the effect of forward orientation and reverse orientation on the immunoassays of this invention, an immunoassay for TNF-alpha was run in forward and reverse formats. In an exemplary forward two site assay, such as a forward two-site immuno-PCR assay, reporter antibody is incubated with sample for a set time, e.g., 2 hours, followed by incubation with the capture antibody bound to paramagnetic particles. In the reverse orientation more conventionally used, the sample is incubated with the solid phase antibody bound to the paramagnetic particles, incubated for a set time, e.g., 2 hours, and then the reporter antibody is added to complete the two-site immuno complex.

The study demonstrated that the forward assay format achieved a greater sensitivity than the reverse format.

### Materials:

1. TNF-200 ng/mL in 0.1% BSA/PBS

2. Reporter antibody, anti TNF ab #1 rAb-035 Mono Q purified

3. 1% BSA PBS

4. Wash buffer

5. PCR Mix

6. Capture antibody, Anti TNF MAb

7. PCR Mix

### Assay Procedure:

This experiment was performed in an LC 480 microplate.

TNF stock antigen was diluted in BSA/PBS from 200 pg/mL to 0.02 pg/mL in 10 fold dilutions. Also included were 5, 25 and 100 pg/mL levels.

### Forward Assay Format

25 uL of the sample was added to the wells in the Roche LC 480 96 well microplate.

75 uL of 16.7 pM reporter Ab was added to the sample in each well.

The microplate was placed on a microplate shaker for one minute at 500 rpm to thoroughly mix the reactants and then the plate was incubated an additional 2 hours at room temperature

10 uL of capture particles was added to the sample containing the reporter Ab and allowed to complex with TNF-alpha bound to reporter antibody.

The microplate containing the reaction mixture was incubated on a microplate shaker for 30 minutes at 500 rpm.

The microplate was washed extensively on a Tecan Hydroflex automated plate washer.

100 uL of the PCR mix was added to each well.

The LC 480 microplate was covered with sealing foil and centrifuged for 1 minute at 500×g.

The LC 480 microplate was placed in the Light Cycler 480II (LC 480II) (Roche Diagnostics) and RT PCR was performed.

### Reverse Assay Format

25 uL sample was added to the wells in the Roche LC 480 96 well microplate.

10 uL of capture particles were added to sample.

The microplate was incubated on a microplate shaker for 2 hours at 500 rpm.

75 uL of the 16.7 pM rAb was added to the sample and the capture particles suspension.

The microplate was incubated on a microplate shaker for 30 minutes at 500 rpm.

The microplate was washed extensively on a Tecan Hydroflex automated plate washer.

100 uL of the PCR mix was added to each well.

The LC 480 microplate was covered with the sealing foil and centrifuged for 1 minute at 500×g.

The LC 480 microplate was placed in the LC 480II and RT PCR was performed.

### Results:

This study was performed according to the protocol as outlined above with TNF alpha concentration ranges of between 0.625 pg/mL to 10 pg/mL. The results are shown in Table 9.

TABLE 9
Summary of Results

| | Cycle Counts | | | | ΔCp 0 Difference | |
|---|---|---|---|---|---|---|
| Format | Forward | | Reverse | | (Forward − | |
| Conc | Cp | ΔCp 0 | Cp | ΔCp 0 | Reverse) | |
| 0 | 26.35 | 0.00 | 26.37 | 0.00 | −0.02 | |
| 0.625 | 23.39 | 2.96 | 25.50 | 0.87 | −2.11 | |
| 1.25 | 22.87 | 3.49 | 25.09 | 1.28 | −2.22 | |
| 2.5 | 22.03 | 4.32 | 24.40 | 1.97 | −2.37 | |
| 5 | 21.03 | 5.32 | 23.48 | 2.89 | −2.44 | |
| 10 | 19.89 | 6.46 | 22.80 | 3.57 | −2.91 | |
| | | | | | −2.41 | Avg |

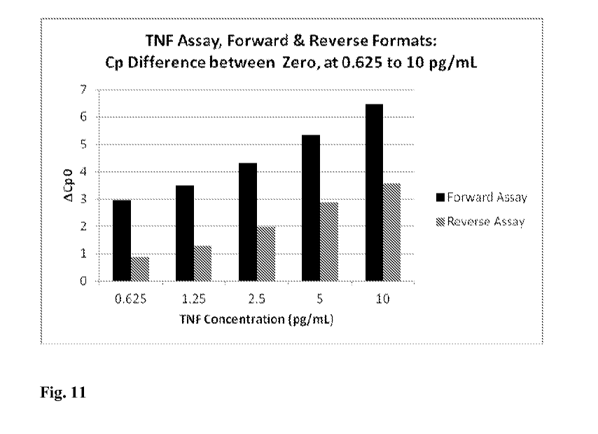

The results indicated that the forward TNF alpha assay format has approximately 5 fold more sensitivity than the reverse assay format for this particular TNF alpha concentration range as demonstrated in FIG. 11. The forward format shows larger DCp 0 (better signal-to-noise ratio) values than the reverse format at the indicated concentrations. The average delta value is a difference of 2.4 cycles.

### Example 5

### PSA Assay

### Example 5A

### Determination of Optimal Concentration for the Reporter Antibody-DNA Conjugate for PSA Detection by Immuno-PCR

In an exemplary immuno-PCR assay for PSA, immuno-PCR assays for [PSA] were performed. Samples each containing PSA at a concentration of 0 and 2.5 pg/ml in 100 ul PBS containing 1% BSA, 0.1% salmon sperm DNA and 0.1% sodium azide were contacted with various concentrations of anti PSA MAb labeled with double stranded oligonucleotide. The sample was incubated with reporter antibody for 2 hours at room temperature. Assays were performed in triplicate.

### Production of Signal Nucleic Acid-Anti-PSA Conjugate

The first antibody is conjugated (chemically linked) to an oligonucleotide of 60 bases. This reporter antibody is then diluted in a buffered diluent containing bovine serum albumin (BSA) and a surfactant to decrease non-specific binding at a pH range of 7.0-7.5.

Prostate-specific antigen (PSA) and sandwich-paired monoclonal antibodies were obtained from BiosPacific Inc. (Emeryville, Calif.). Oligonucleotides of 60 bases were synthesized to contain a functional amine attached to the 5′ end through a 12-carbon spacer arm from Glen Research Corp. (Sterling, Va.) and purified by preparative polyacrylamide gel electrophoresis. The 5′ amino function was activated with a 100-fold excess of disuccinimidyl suberate to minimize cross-linking. The intermediate was rapidly purified by gel-filtration fast protein liquid chromatography (FPLC) in 5 mmol sodium citrate (pH 5.4) in order to maintain the second succimidyl function. The DNA was concentrated by centrifugal ultrafiltration at 4° C. and combined immediately at room temperature with 10 mg/ml antibody in 0.3 mol phosphate buffer (pH 8) and 0.45 mol NaCl for 1 hour. Unreacted antibody was removed by size-exclusion FPLC using a Superose S-200 column from GE Healthcare (Piscataway, N.J.) that had been equilibrated in Tris-buffered saline (pH 7.4). Unreacted oligonucleotide was removed by anion-exchange FPLC using a Mono Q column from GE Healthcare and 5%/min salt-gradient elution to 1 mol in 20 mmol Tris (pH 7.4). Typically, 50% of the protein was recovered as conjugate.

Both native and sodium dodecyl sulfate (SDS) gel electrophoresis revealed the presence of antibody containing predominantly one or two strands of the 60-mer. The presence of covalent antibody does not interfere with PCR signal. Likewise, the DNA label does not obstruct binding to antigen, as determined by HRP-labeled second-antibody detection of solid-phase antigen.

An oligonucleotide having the following sequence was used to form the DNA-reporter antibody conjugate:

Reporter Sequence:
(SEQ ID NO: 1)
5′-{C12 NH2}TGCGTAGCGATGACTAGCTGCTGATCGATATTAGCTAG
CATCAGCGATCGATACGAGCA-3′

An oligonucleotide having the following sequence is complementary to the reporter sequence, and hybridizes with the reporter antibody-reporter sequence conjugate to form a double stranded hybrid attached to the reporter antibody.

Reporter Sequence Complement:
(SEQ ID NO: 2)
5′-TGCTCGTATCGATCGCTGATGCTAGCTAATATCGATCAGCAGCTAGT
CATCGCTACGCA-3′

The reporter DNA can be amplified by PCR using the following primers:

Primer 1 Sequence:
(SEQ ID NO: 3)
5′-TGCGTAGCGATGACTAGCTGCTG-3′
Primer 2 Sequence:
(SEQ ID NO: 4)
5′-TGCTCGTATCGATCGCTGATGCT

### Production of Capture Nucleic Acid-Anti-PSA Conjugate

The second antibody is immobilized on a streptavidin-coated para-magnetic particle of approximately 1 micron in diameter. The capture antibody has biotin chemically attached to it, using EZ-Link Sulfo-NHS-LC-Biotin (Sulfosuccinimidyl-6-(biotinamido) hexanoate, catalog number 21335, in accordance with the manufacturer's instructions. The antibody is subsequently coated onto the para-magnetic particle through a streptavidin linker.

Five micrograms of Seradyn streptavidin paramagnetic microparticles coated with biotin-labeled second MAb were added to each sample containing reporter antibody-analyte complexes with gentle agitation, and incubated for 30 minutes at room temperature.

The micro particles were washed 5 times by repeated magnetic collection and resuspension with 200 ul volumes of 0.1M Tris, 50 mM KCl, 2 mM MgCl2, 0.05% Tween-20, pH 7.2. The wash steps typically take 5-10 min each, which duration comprises incubation and handling steps Following the last wash, the particles were resuspended in qPCR mix containing Platinium Taq, primers, 10 uM fluorescein, SYBR Green and subjected to real time PCR using a Bio-Rad I-Cycler programmed for 40 cycles of 15 sec 95 degree denaturation, and 1 min 62 degree extension conditions.

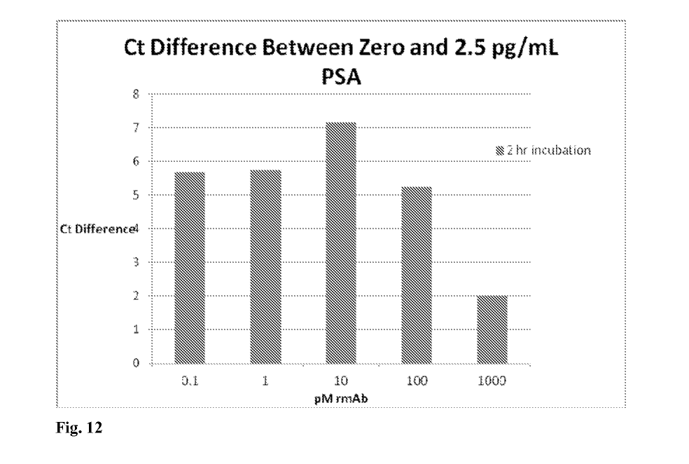

FIG. 12 shows a graph of the results of the PSA assay, which is shown in tabular form below, in Table 10. The data is expressed in threshold cycle difference between the blank (zero PSA) and the PSA sample for each concentration of MAb-DNA conjugate reactant. Surprisingly, the assays using reporter antibody concentrations of 0.1, 1.0, and 10.0 pM reporter antibody to PSA gave higher signal above background levels than the assay using 100 pM or 1000 pM reporter antibody. In one embodiment, the optimal signal to noise is obtained with an input concentration of 10 pM MAb-DNA.

TABLE 10
2 hr incubation

| | Zero | | |
|---|---|---|---|
| rmAb-DNA (pM) | PSA | 2.5 pg/mL PSA | Difference |
| 0.1 | 30 | 24.33 | 5.67 |
| 1 | 27 | 21.27 | 5.73 |
| 10 | 25.73 | 18.56 | 7.17 |
| 100 | 23.15 | 17.91 | 5.24 |
| 1000 | 19.11 | 17.1 | 2.01 |

Determination of k_{d} Values

Multiple antibodies to PSA are obtained. The K_{d}, kₐ, and k_{d} values for each antibody are obtained using any known method, such as using a Biacore system. Each of the antibodies with a K_{d}, value of less than about 1×10⁻⁸ M is used as a reporter antibody in an immuno-PCR assay as described below. The concentration of each reporter antibody is the same in each set of assays, and that concentration may be 0.1, 1.0, or 10.0 pM.

### Assay Procedure

The reporter MAb-DNA conjugate was reacted with sample in a microtiter plate format to form a first immune complex with PSA. The immune complex was then captured onto paramagnetic microparticles coated with the second capture MAb, forming an insoluble sandwich immune complex. The microparticles were washed by several cycles of magnetic capture and re-suspension to remove excess reporter MAb-DNA conjugate.

The specifically bound DNA label was then detected by subjecting suspended particles to PCR conditions and monitoring the generations of amplicon in real time. Quantitation of PSA was achieved by monitoring the number of PCR thermocycles it takes to generate a predetermined fluorescent signal over baseline. This was accomplished by automatic instrumentation designed to monitor fluorescence intensity as a function of cycle number. The amount of DNA initially present in the sample was inversely proportional to the threshold cycle (Ct). PSA concentrations (pg/mL) of the samples were calculated from the calibration curve.

A 20 μL sample was placed in a well of a 96-well microtiter plate, followed by 75 μL of MAb-DNA to final concentration of MAb-DNA as indicated. The plate was securely covered, placed on a platform shaker and gently agitated at approximately 500 rpm for one minute to thoroughly mix reactants. The reaction was allowed to proceed for an additional two hours at room temperature without mixing. The plate was uncovered and 10 μL (5 ug) of a suspension of paramagnetic micro particles coated with second capturing antibody is added. The 96 well plate was covered with another seal and placed on a platform shaker at 500 rpm and incubated for 30 minutes at room temperature.

The 96-well plate was placed onto a magnetic rack and four sequential wash and separation steps are performed using a 125 μL wash solution composed of a tris buffered solution of normal saline with 0.05% sodium and 0.5% Tween-20, pH 7.4. The plate was centrifuged at 450-500×g for one minute. A wash procedure using an automated micro plate washer is also suitable.

### PCR Reagent Addition Step

30 μL of PCR Reagent was added to each well. The plate was covered with an optical adhesive cover then centrifuged at 450-500×g for one minute at a minimal brake setting.

Detection was accomplished by performing 35 cycles of qPCR. Any other commercially available device for carrying out real-time PCR may also be used.

The concentration of PSA was determined by comparison and extrapolation from appropriate control samples using the threshold cycle.

In certain embodiments, the exemplary PSA assays disclosed herein are designed for patients whose first serum sample is collected at 6 weeks after radical prostatectomy and whose total PSA value is at or below 0.1 ng/mL, preferably as determined by a PSA assay approved or cleared by the FDA. In certain further embodiments, the assays of this invention are used to determine the rate of change of serum total prostate specific antigen over a period of time in pg/mL per month (e.g., slope, Ln of the slope, or doubling time). U.S. Application No. 20090246781, incorporated herein by reference in its entirety, describes the use of [PSA] cut-offs and/or velocity of change in [PSA] for determining stable disease or recurrence of prostate cancer following radical prostatectomy. In one embodiment, the slope is indicated for use as a prognostic marker in conjunction with clinical evaluation as an aid in identifying those patients at reduced risk for recurrence of prostate cancer for the eight year period following prostatectomy.

### Patient Sample Collection and Testing Schedule

In one embodiment, results are calculated as the linear slope of three total PSA test results obtained on three serum samples collected between six weeks and 20 months post-radical prostatectomy. In a further embodiment, all three samples from a single patient are tested in a single assay run. In another embodiment, the first serum sample for the assay is collected at least six weeks after the date of radical prostatectomy, frozen at −70° C. and stored at −70° C.

In certain embodiments, the date of radical prostatectomy is sent to the laboratory along with the first sample. In a further embodiment, the date of sample collection is sent to the laboratory with each sample. In a further embodiment, the second serum sample collection date should be at least two months after the first sample collection date. In another embodiment, the third serum sample collection should be completed within 10 to 20 months (no sooner than 10 months) of the date of radical prostatectomy, and at least two months after the second sample. In certain other embodiments, the first and second serum samples are not tested by the exemplary assay at the time of collection. Rather, the two samples are stored at ≦−70° C. until the third sample is available for testing. Frozen samples are stable for at least 20 months. All three samples are tested in the same assay run. In another embodiment, dates of sample collection are entered into a software program to ensure that the requirements for the proper time intervals for sample collection are met.

### Example 6

### Comparison of Reverse and Forward Assay Formats Using a Low Concentration Reporter Antibody

To study the effect of forward orientation and reverse orientation on the immunoassays of this invention, an immunoassay for PSA was run in forward and reverse formats. In a forward two site assay, such as a forward two-site immuno-PCR assay, reporter antibody is incubated with sample for a set time, e.g., 2 hours, followed by incubation with the capture antibody bound to paramagnetic particles. In the reverse orientation more conventionally used, the sample is incubated with the solid phase antibody bound to the paramagnetic particles, incubated for a set time, e.g., 2 hours, and then the reporter antibody is added to complete the two-site immuno complex. Materials used in the assay are noted below.

### Materials:

1. Serum based Assay diluent

1. 5 pg/mL PSA Calibrator in diluent

2. 25 pg/mL PSA Calibrator in diluent

3. 100 pg/mL PSA Calibrator in diluent

4. QC Low Control˜2 pg/mL PSA

5. QC Mid Control˜7 pg/mL PSA

6. QC High Control˜60 pg/mL PSA

7. Reporter antibody 12.5 pM

8. Capture Ab on paramagnetic particles

9. PCR Mix

### Methods:

Forward Assay Format
1. 20 uL sample was added to wells in a Roche LC 480 96 well microplate.
2. 75 uL of 12.5 pM reporter antibody (rAb) was added to the sample for a final concentration of 9.9 pM.
3. The microplate was placed on a micoroplate shaker for r 1 minute at 500 rpm to thoroughly mix reactants and incubated an additional 2 hours.
4. 10 uL of capture particles were added to the sample and rAb solution.
5. The microplate was incubated on a microplate shaker for 30 minutes at 500 rpm.
6. The microplate was washed extensively on a Tecan Hydroflex automated plate washer.
7. 100 uL of PCR mix was added to each well.
8. The LC 480 microplate was covered with Roche sealing foil and centrifuged for 1 minute at 500×g.
9. The LC 480 microplate was placed in the LC 480II and RT PCR was performed.

Reverse Assay Format
1. 20 uL sample was added to wells in Roche LC 480 96 well microplate.
2. 10 uL of capture antibody particles was added to the sample.
3. The microplate was incubated on a microplate shaker for 2 hours at 500 rpm.
4. 75 uL of 12.5 pM rAb was added to sample and capture particles suspension.
5. The microplate was incubated on a microplate shaker for 30 minutes at 500 rpm.
6. The microplate was washed extensively on a Tecan Hydroflex automated plate washer.
7. 100 uL of PCR mix was added to each well.
8. The LC 480 microplate was covered with Roche sealing foil and centrifuged for 1 minute at 500×g.
9. The LC 480 microplate was placed in the LC 480II and RT PCR was performed.

TABLE 11
Results from the exemplary forward and reverse assays
PSA Forward vs Reverse Assay
Summary Table

| Cycle Counts | |
|---|---|
| Forward Assay | Reverse |

| [PSA] | Avg Ct | ΔCp 0 | Avg Ct | ΔCp 0 |
|---|---|---|---|---|
| 0 | 25.75 | 0.00 | 26.63 | 0.00 |
| 5 | 19.68 | 6.07 | 23.08 | 3.55 |
| 25 | 17.29 | 8.46 | 20.89 | 5.74 |
| 100 | 15.23 | 10.52 | 19.28 | 7.35 |
| | Avg Ct | pg/mL | Avg Ct | pg/mL |
| QC High | 16.09 | 56.37 | 19.81 | 63.40 |
| QC Low | 20.95 | 2.13 | 24.26 | 1.94 |
| QC MAS | 19.07 | 7.57 | 22.85 | 5.79 |
| PCR zero | 30 | N/A | 30 | N/A |
| Slope | −3.42 | | −2.92 | |
| Intercept | 22.07 | | 25.08 | |

The results show that while assays in both formats provided approximately the same pg/mL readback on the zero PSA control samples, the forward assay had an almost ideal slope for the PSA calibrators while the reverse assay slope was significantly lower than the ideal value of 3.32. Most importantly, the signal to noise ratios (Δ Cp 0) for the forward assay is surprisingly approximately ten fold superior to those of the reverse assay, when a 10 pM concentration of reporter antibody is used in this exemplary assay of the invention.

### Example 7

### Kits

In one aspect, the instant disclosure also provides a kit comprising specificity molecule reagents set, PCR reagent and calibrator set. In some embodiments the kit may alternatively include software and/or directions for use. The kit may include any one of, or combination of, the components listed above. In some embodiments, the specificity molecule reagent set may be an Antibody Reagent Set comprising reporter antibodies as disclosed in this invention, capture antibodies conjugated to a solid support. The kit may further comprise reagents and/or a calibrator set. When stored and handled as directed, reagents are stable until the expiration date printed on the reagent vial labels and the kit labels.

The kit may also, in some embodiments, provide instructions for the collection of patient samples. For an analyte for which multiple samples will be tested over time, the collection of patient samples will occur at multiple time points. As an example, where the analyte is PSA, the collection of patient samples will occur at three time points between 6 weeks and 20 months post-radical prostatectomy for each patient. The Software provides quality control support to the sample collection process.

In one embodiment, the multiple samples from a given patient are preferably tested for the analyte in a single assay run, and preferably on one sample holder or plate. In one embodiment the analyte testing will not be performed until all the samples from a patient have been collected over the desired time course.

In certain embodiments, the antibody reagent set comprises the following:

| | | |
|---|---|---|
| Target Capture | Contains two tubes, | Biotinylated monoclonal anti-analyte |
| Reagent | 0.96 mL each. | murine antibody attached to streptavidin- |
| | Store at 2-8° C. until | coated paramagnetic microparticles, |
| | expiration date. | buffers, salts, surfactant and 0.09% sodium |
| | | azide. |
| Reporter Antibody | Contains two bottles, | Monoclonal analyte-specific murine |
| Reagent | 7.2 mL each. | antibody labeled with reporter DNA |
| | Store at 2-8° C. until | sequence, bovine serum albumin, murine |
| | expiration date. | IgG, buffers, salts, surfactant and 0.09% |
| | | sodium azide. |

The antibodies may be murine, humanized, camel, goat, sheep, rabbit, or from any other source known in the art and as further disclosed herein. Where PSA is the analyte, the reagents are as follows:

| | | |
|---|---|---|
| Target Capture | Contains two tubes, | Biotinylated monoclonal anti-PSA murine |
| Reagent | 0.96 mL each. | antibody attached to streptavidin-coated |
| | Store at 2-8° C. until | paramagnetic microparticles, buffers, salts, |
| | expiration date. | surfactant and 0.09% sodium azide. |
| Reporter Antibody | Contains two bottles, | Monoclonal analyte-specific PSA antibody |
| Reagent | 7.2 mL each. | labeled with reporter DNA sequence, |
| | Store at 2-8° C. until | bovine serum albumin, murine IgG, |
| | expiration date. | buffers, salts, surfactant and 0.09% sodium |
| | | azide. |

In certain embodiments, using PSA as the exemplary analyte, the PCR Reagent and Calibrator Set comprises the following:

| | | |
|---|---|---|
| PCR Reagent | Contains two | Taq DNA polymerase, dNTP's, buffer, salts, |
| | bottles, 2.88 mL | SYBR ® Green I, primers specific to the |
| | each. | reporter DNA, reference dye and surfactant. |
| | Store at −10 to −30° C. | |
| | until | |
| | expiration date. | |
| Analyte Calibrators, | Contains two vials, | Purified human analyte (e.g., prostate specific |
| e.g., PSA | 0.06 mL of each. | antigen (90% PSA-ACT + 10% Free PSA)), |
| Calibrators | Store at −10 to −30° C. | Equine serum, stabilizer and 0.09% sodium |
| 5, 25 and 100 pg/mL | until | azide. |
| | expiration date. | |

In certain embodiments, using PSA as the exemplary analyte, the Software and Directions for Use comprises the following:

| | |
|---|---|
| Software (e.g., | Contains ProsVue |
| ProsVue) | Software CD-ROM, |
| | software DFU and |
| | ProsVue DFU |
| | Store at 15 to 30° C. |

Certain individuals have antibodies to mouse protein which can cause interference in immunoassays that employ antibodies derived from mice. In certain embodiments, the assay reagents disclosed herein may contain additives to reduce the probability of such an occurrence but rare extremely high levels of such antibodies may interfere with the assay. This is a greater probability with patients who have been treated with preparations of mouse monoclonal antibodies for diagnosis or therapy. Results should be interpreted with caution for such patients.

It is critical when using a nucleic acid label and PCR detection that caution be used when handling the Reporter Specificity Molecule Reagent to avoid this material contaminating any laboratory equipment that will be used in the wash and PCR amplification phases of the assay procedure. The signal used to measure the analyte concentration is associated with the Reporter Reagent.

Kits may also alternatively contain one or more of the following:
1. Wash Solution Kit, REF 800-8002 (IRIS buffered saline with surfactant).
2. Sample Diluent Kit, REF 800-8003 (Buffered serum based diluent with preservative).
3. Control Kit, REF 800-8001 (approximately 2, 8, and 80 pg/mL of PSA in serum based diluent).
4. Applied Biosystems® (AB) 96-well MicroAmp plate, part #4346906.
5. Corning plate sealers, part #3095, or equivalent.
6. AB optical adhesive cover, part #4360954.
7. Magnetic bead separator block for 96 well PCR plates.

### Example 8

### Specimen Collection and Preparation

In one embodiment, the test sample type is human serum. In one embodiment, blood is drawn by standard phlebotomy techniques and collected into a red-top tube or gel barrier tube. In one embodiment, specimens is collected in such a way as to avoid hemolysis. In one embodiment, specimens are allowed to clot fully and the serum is separated by centrifugation. In one embodiment, residual fibrin and cellular matter are removed prior to analysis. In one embodiment, serum that is turbid or contains particulate matter are centrifuged before assay. In one embodiment, the first and second assay test samples are frozen at ≦−70° C. until the third sample is available for testing. In one embodiment, assay samples are stored at 2-8° C. for up to 48 hours prior to freezing or testing, but are stored at ≦−70° C. if held for longer periods (Woodrum D., 1998²⁷). In one embodiment, samples that have been frozen are mixed thoroughly after thawing by low speed vortexing or by gently inverting followed by centrifugation. In a further embodiment, vigorous agitation of serum samples are avoided as this can result in foam formation.

In one embodiment, samples are run in duplicate and the average of these two values is the reportable patient result. If the percent coefficient of variation (% CV) of the duplicate results exceeds 20%, reanalyze the patient sample in duplicate and report the average of the duplicate results.

### Example 9

### Reagent Preparation for Assay

In one embodiment, Target Capture Reagent, Reporter Reagent, and PCR Reagent are brought to room temperature just prior to use. Accelerated warming at temperatures exceeding 37° C. is not recommended. Target capture reagent is always be mixed by gentle vortexing just prior to use. In one embodiment, the Calibrators are brought to room temperature just prior to use and mixed by gentle vortexing. The assay may be carried as above.

### Example 10

### Determination of Results for Analytes Tested Multiple Times

The results are calculated using the Assay Set-Up and Analysis portion of the software. Briefly, raw Ct results are generated by the AB 7500 Fast Dx PCR Sequence Detection Software (SDS) version 1.4. Using Ct values from the calibrators, a three-point linear fit mathematical model is employed. The concentration in the sample (in pg/mL) is mathematically determined from the Ct by using the calculation method below:

The straight line equation y=mx+b is employed to calculate log 10 analyte levels (x) where y equals Ct values of the calibrators, m is the slope (Ct/log₁₀ pg/mL analyte) and b is the y intercept when log₁₀ pg/mL analyte is zero.

Concentrations for the unknowns are determined from the following equation:

PSA (pg/mL)=antilog₁₀ of (Ct of unknown/slope)−(intercept/slope)

### Determination of the Slope

The software determines the slope by linear regression using the least squares method in the following formula:

$$\frac{{\sum }_{i = 1}^{3} ( {x}_{i} - \overline{x} ) ( {y}_{i} - \overline{y} )}{{\sum }_{i = 1}^{3} {( {x}_{i} - \overline{x} )}^{2}}$$

Where y=pg/mL and x=days between samples. The slope in pg/ml/day is then converted to pg/mL/month by multiplying by 30.4375 (average days/month). No slope is calculated if any value is less than 0.65 pg/mL.

### Interpretation and Reporting of Results

In one embodiment where PSA is the analyte the software categorizes patients as “at reduced risk for prostate cancer recurrence” or “not at reduced risk for prostate cancer recurrence” based on the slope criteria listed in the table below. The software reports results as the slope (pg/mL per month) and categorization.

| Risk Category | Slope |
|---|---|
| At reduced risk for prostate | Less than or equal to 2.0 pg/mL |
| cancer recurrence | per month |
| Not at reduced risk for prostate | Greater than 2.0 pg/mL per month |
| cancer recurrence | |

### Example 11

### Determination of Interference by Other Substances in the Sample

For assays which may be subject to interference by other blood constituents, other components known to be present in blood are tested for interference with the assay. The substances may include one or more or all of the following potential interfering substances and/or any combination of two or more of the substances:

| Potentially interfering substances | | |
|---|---|---|
| Bilirubin, conjugated | 30 | mg/dL |
| Cholesterol | 500 | mg/dL |
| Creatinine | 5.0 | mg/dL |
| Hemoglobin | 200 | mg/dL |
| Immunoglobulin G | 6 | g/dL |
| Triglycerides | 1000 | mg/dL |
| Urea | 260 | mg/dL |
| Uric acid | 23.5 | mg/dL |

| Interference by drugs | | |
|---|---|---|
| 10-hydroxynortriptyline | 700 | ng/mL |
| 5-Fluorouracil | 390 | μg/mL |
| Acetaminophen | 200 | μg/mL |
| Ampicillin | 53 | μg/mL |
| Ascorbic acid | 60 | μg/mL |
| Biotin | 50 | ng/mL |
| Caffeine | 60 | μg/mL |
| Carbamazepine | 30 | μg/mL |
| Chloramphenicol | 50 | μg/mL |
| Cimetidine | 20 | μg/mL |
| Ciprofloxacin | 10 | μg/mL |
| Cisplatin | 12 | μg/mL |
| Cotinine | 1.9 | μg/mL |
| Cyclophosphamide | 375 | μg/mL |
| Dextran-40 | 60 | mg/mL |
| Digoxin | 6.1 | ng/mL |
| Doxorubicin | 240 | ng/mL |
| Erythromycin | 60 | μg/mL |
| Ethanol | 4 | mg/mL |
| Ethosuximide | 250 | μg/mL |
| Flutamide | 500 | ng/mL |
| Furosemide | 60 | μg/mL |
| Gentamicin | 10 | μg/mL |
| Heparin sodium | 3 | U/mL |
| Ibuprofen | 500 | μg/mL |
| Leuprolide acetate | 200 | ng/mL |
| Lidocaine | 12 | μg/mL |
| Lithium | 22.5 | μg/mL |
| Methotrexate | 910 | μg/mL |
| Paclitaxel | 6.5 | μg/mL |
| Pamidronate | 9 | μg/mL |
| Phenytoin | 50 | μg/mL |
| Prednisone | 300 | ng/mL |
| Primidone | 40 | μg/mL |
| Prochlorperazine | 1 | μg/mL |
| Salicylic acid | 600 | μg/mL |
| Sulfamethoxazole | 400 | μg/mL |
| Tamoxifen | 1.5 | μg/mL |
| Trimethoprim | 40 | μg/mL |
| Valproate sodium | 500 | μg/mL |
| Vancomycin | 100 | μg/mL |
| Vinorelbine | 1.2 | μg/mL |

### Example 12

### High Dose Hook Effect

Patient samples with high levels of analyte such as PSA levels can cause a paradoxical decrease in the signal reported. In the assay of this invention, because of the robustness and high dynamic range, samples with analyte concentrations as high as 50,000 pg/mL do not exhibit this effect. To accurately quantitate a value >100 pg/mL, samples can be diluted accordingly to a value in the assay's reportable range and re-assay.

To determine linearity or precision, high and low concentration analyte samples are mixed at varying proportions to mimic a range of concentration of analyte. The concentration of analyte over which measurement is linear with less than 25%, 24, 23, 22, 21, 20, 19, 18, 17, 16, 15, 14, 13, 12, 11, 10, 9, 8, 7, 6, or 5% deviation is then determined. For example, high and low concentration samples of PSA may be mixed at varying proportions to mimic a range of concentration of PSA typically observed post-prostatectomy. For total PSA, the method has been demonstrated to be linear from 0.65 to 100 pg/mL with deviation from linearity less than 24% (final rAB level was approximately 10 pM).

### Example 13

### Sensitivity and Measuring Interval

Four low level analyte samples are prepared (0.55, 0.65, 0.75 and 1.0 pg/mL) by adding analyte standard to the Sample Diluent. Sixty replicates of each sample and Sample Diluent are run in the assay. Limit of blank (LOB), limit of detection (LOD), and limit of quantitation (LOQ) are determined in accordance with the CLSI EP17-A requirements. In one embodiment:

The LOB is the highest measurement result that is likely to be observed (with a probability [alpha] of 0.05 [5%]) for a blank sample.

The LOD is the lowest amount of analyte in a sample that can be detected with type I and II error rates set to 5%.

The limit of quantitation (LOQ) is the lowest amount of analyte in a sample that can be reliably detected and which meets the pre-specified requirements for accuracy (80-120%) and precision (<25%).

In one embodiment, four low level PSA samples are prepared (0.55, 0.65, 0.75 and 1.0 pg/mL), and the LOB, LOD and LOQ are determined.

### Equimolarity

To demonstrate equimolarity in an PSA assay (i.e., that the assay recognizes free PSA and PSA-ACT equally well), sets of samples with free PSA concentrations ranging from zero to 100 percent and total PSA concentrations of 5, 30, 70 and 100 pg/mL are assayed, which demonstrates that the assay can recognize free PSA and PSA-ACT equally well.

All patents, patent applications and publications cited in this application are hereby incorporated by reference in their entirety for all purposes to the same extent as if each individual patent, patent application or publication were so individually denoted.

Although certain embodiments and examples have been described in detail above, those having ordinary skill in the art will clearly understand that many modifications are possible in the embodiments and examples without departing from the teachings thereof. All such modifications are intended to be encompassed within the below claims of the invention.

### BIBLIOGRAPHY

1. Bjerner J, Nustad A, Norum L F, Olsen K H, Bormer O P (2002) Immunometric assay interference: Incidence and prevention. Clin Chem 48: 613-21.
2. Collins G N, Martin P J, Wynn-Davies A, Brooman P J, O'Reilly P H (1997) The effect of digital rectal examination, flexible cystoscopy and prostatic biopsy on free and total prostate specific antigen and the free-to-total prostate specific antigen ratio in clinical practice. J Urol 157: 1744-7.
3. Cooperberg M R, Broering J M, Litwin M S, et al (2004) The contemporary management of prostate cancer in the United States: Lessons from the Cancer of the Prostate Strategic Urological Research Endeavor (CaPSURE), a national disease registry. J Urol 171: 1393-1401.
4. D'Amico A V, Moul J W, Carroll P R, Sun L, Lubeck D, Chen M H (2003) Surrogate marker for prostate cancer-specific mortality following radical prostatectomy or radiation therapy. J Natl Cancer Inst 95: 1376-83.
5. DHH-S(NIOSH) Publication No. 78-127 (August 1976) “Current Intelligence Bulletin 13—Explosive Azide Hazard.” Available online at http://www˜cdc.gov/niosh.
6. Dotan Z A, Bianco Jr F J, Rabbani F, et al (2005) Pattern of prostate-specific antigen (PSA) failure dictates the probability of a positive bone scan in patients with an increasing PSA after radical prostatectomy. J Clin Oncol 23: 1962-8.
7. Freedland S J, Humphreys E B, Mangold L A, et al (2007) Death in patients with recurrent prostate cancer after radical prostatectomy: Prostate-specific antigen doubling time subgroups and their associated contribution to all-cause mortality. J Clin Oncol 25: 1765-71.
8. Hendrickson E R, Truby T M, Joerger R D, Majarian W R, Ebersole R C (1995) High sensitivity multianalyte immunoassay using covalent DNA-labeled antibodies and polymerase chain reaction. Nucleic Acids Res 23: 522-9.
9. Jablonski E, Adams T A (2006) The merging of nucleic acid detection and immunoassays. IVD Technology, pp. 63-9, November 2006.
10. Joerger R, Truby T M, Hendrickson E R, et al. Analyte detection with DNA-labeled antibodies and polymerase chain reaction. Clin Chem 41: 1371-7.
11. Kattan M W, Wheeler T M, Scardino P T (1999) Postoperative nomogram for disease recurrence after radical prostatectomy for prostate cancer. J Clin Oncol 17: 1499-1507.
12. Kricka, L J. Interferences in immunoassays—still a threat (2002) Clin Chem 46:1037-8.
13. May F, Hartung R, Breul J (2001) The ability of the American Joint Committee on Cancer Staging system to predict progression-free survival after radical prostatectomy. BJU Int 88: 702-7.
14. Morgan W R, Zincke H, Rainwater L M, Myers R P, Klee G G. Prostate specific antigen values after radical retropubic prostatectomy for adenocarcinoma of the prostate: Impact of adjuvant treatment (hormonal and radiation). J Urol 145: 319-23, 1991.
15. Moul J W (2000) Prostate specific antigen only progression of prostate cancer. J Urol 163: 1632-42.
16. Mullis K B, Faloona F A (1987) Specific synthesis of DNA in vitro via a polymerase-catalyzed chain reaction. Methods Enzymol 155: 335-51.
17. Partin A W, Pound C R, Clemens J Q, Epstein J I, Wash P C (1993) Serum PSA after anatomical radical prostatectomy: the Johns Hopkins experience after 10 years. Urol Clin North Am 20: 713-25.
18. Partin A W, Pearson J D, Landis P K, et al (1994a) Evaluation of serum prostate—specific antigen velocity after radical prostatectomy to distinguish local recurrence from distant metastases. Urology 43: 649-59.
19. Partin A W, Kattan M W, Subong E N P, et al (1997) Combination of prostate-specific antigen, clinical stage, and Gleason score to predict pathological stage in men with localized prostate cancer: A multi-institutional update. JAMA 277: 1445-51.
20. Pound C R, Partin A W, Eisenberger M A, et al (1999) Natural history of progression after PSA elevation following radical prostatectomy. JAMA 281: 1591-7.
21. Rafferty B, Rigsby P, Rose M, et al (2000) Reference reagents for prostate-specific antigen (PSA): establishment of the First International Standards for free PSA and PSA (90:10). Clin Chem 46: 1310-7.
22. Sano T, Smith C L, Cantor C R (1992) Immuno-PCR: Very sensitive antigen detection by means of specific antibody-DNA conjugates. Science 258: 120-2.
23. Shariat S F, Kattan M W, Vickers A J, Karakiewicz P I, Scardino P T (2009) Critical review of prostate cancer predictive tools. Future Oncology 5: 1555-84.
24. Soergel T M, Koch M O, Foster R S, et al (2001) Accuracy of predicting long-term prostate specific antigen outcome based on early prostate specific antigen recurrence results after radical prostatectomy. J Urol 166: 2198-2201.
25. Thompson I M, Goodman P J, Tangen C M, Lucia M S, Miller G J, et al (2003) The influence of finasteride on the development of prostate cancer N Engl J Med 349: 215-24.
26. Tollefson M K, Slezak J M, Liebovich B C, Zincke H, Blute M L (2007) Stratification of patient risk based on prostate-specific antigen doubling time after radical retropubic prostatectomy. Mayo Clin Proc 82: 422-7.
27. Woodrum D, York L (1998) Two-year stability of free and total PSA in frozen serum samples. Urology 52: 247-51.

What is claimed is:

1. A method for detecting a non-nucleic acid analyte present in a sample to be tested using a forward two-site immuno-PCR assay, the method comprising the steps of:
(1) contacting the sample containing the analyte with:
(a) a reporter conjugate present at a concentration ranging from about 1 to 15 pM or from about 0.15 to 2.25 ng/mL, wherein the reporter conjugate comprises:
(i) a reporter monoclonal antibody capable of specifically binding the analyte in the test sample with a dissociation rate constant lower than about 3.0×10⁻⁴ sec⁻¹; and
(ii) a nucleic acid marker;
thereby forming a first immune complex; and
(2) contacting the first immune complex with a capture monoclonal antibody bound to a solid support, where the second antibody is capable of specifically binding to the analyte in the first immune complex, thereby forming a two-site immune complex bound to the support;
(3) washing the two-site immune complex bound to the support; and
(4) detecting the presence and amount of the nucleic acid marker in the two-site immune complex, wherein the assay detects analyte present in the sample at concentrations of less than 10 picogram/ml.

2. The method of claim 1 wherein the reporter conjugate is present at a concentration ranging from about 0.1 to 10 pM or from about 0.015 ng/mL to 1.5 ng/mL of antibody protein.

3. The method of claim 1 wherein the reporter conjugate is present at a concentration ranging from about 0.1 to 1.0 pM or from about 0.015 ng/mL to 0.15 ng/mL of antibody protein

4. The method of claim 1 wherein the reporter conjugate is present at a concentration ranging from about 3 to 10 pM or from about 0.45 to 1.5 ng/mL of antibody protein.

5. The method of claim 1 wherein the reporter conjugate is present at a concentration ranging from about 3 to 5 pM or from about 0.45 to 0.75 ng/mL of antibody protein.

6. The method of claim 1 wherein the reporter conjugate is present at a concentration ranging from about 5 to 10 pM or from about 0.75 to 1.5 ng/mL of antibody protein.

7. The method of claim 1 wherein the reporter conjugate is present at a concentration ranging from about 10 to 15 pM or from about 1.5 to 2.25 ng/mL of antibody protein.

8. The method of claim 1 wherein the reporter conjugate is present at a concentration of about 0.1 pM or at about 0.015 ng/mL of antibody protein.

9. The method of claim 1 wherein the reporter conjugate is present at a concentration of about 1.0 pM or at about 0.15 ng/mL of antibody protein.

10. The method of claim 1 wherein the reporter conjugate is present at a concentration of about 5.0 pM or at about 0.75 ng/mL of antibody protein.

11. The method of claim 1 wherein the reporter conjugate is present at a concentration of about 3.0 pM or at about 0.45 ng/mL of antibody protein.

12. The method of claim 1 wherein the reporter conjugate is present at a concentration of about 7.5 pM or at about 1.125 ng/mL of antibody protein.

13. The method of claim 1 wherein the reporter conjugate is present at a concentration of about 10 pM or at about 1.5 ng/mL of antibody protein.

14. The method of claim 1 wherein the reporter conjugate is present at a concentration of about 12.5 pM or at about 1.875 ng/mL of antibody protein.

15. The method of claim 1 wherein the reporter conjugate is present at a concentration of about 15 pM or at about 2.25 ng/mL of antibody protein.

16. The method of claim 1 wherein the reporter conjugate is present at a concentration of about 30 pM or at about 4.5 ng/mL of antibody protein.

17. The method of claim 1 wherein the reporter conjugate is present at a concentration of about 90 pM or at about 13.5 ng/mL of antibody protein.

18. The method of claim 1 wherein the solid support is selected from paramagnetic particles, latex or other polymer beads, nanoparticles, glass particles or fibers, or insoluble polysaccharides.

19. The method of claim 1 wherein the solid support comprises paramagnetic particles.

20. The method of claim 1, wherein the detecting further comprises contacting the sample containing the two-site immune complex with reagents and primers for conducting a polymerase chain reaction, and wherein replicating the nucleic acid marker comprises amplifying the nucleic acid marker by the polymerase chain reaction.

21. The method of claim 18, wherein the polymerase chain reaction is a real-time polymerase chain reaction.

22. The method of claim 1, wherein the analyte is HIV p24.

23. The method of claim 1, wherein the analyte is PSA.

24. The method of claim 1, wherein the analyte is TNF-α.

25. The method of claim 1, wherein the analyte is present at less than 10 pg/mL.

26. The method of claim 1, wherein the analyte is present at less than 1.0 pg/mL.

27. The method of claim 1, wherein the analyte is present at less than 0.1 pg/mL.

28. The method of claim 1, wherein the analyte is present at less than 0.01 pg/mL.

29. The method of claim 1, wherein the sample is a serum sample and the limit of quantitation for the assay is about 10 pg/mL.

30. The method of claim 1, wherein the sample is a serum sample and the limit of quantitation for the assay is about 1.0 pg/mL.

31. The method of claim 1, wherein the sample is a serum sample and the limit of quantitation for the assay is about 0.1 pg/mL.

32. The method of claim 1 wherein the reporter conjugate is contacted with the sample containing the analyte for about 90-150 minutes.

33. The method of claim 1 wherein the reporter conjugate is contacted with the sample containing the analyte for about 100-120 minutes.

34. The method of claim 1 wherein the reporter conjugate is contacted with the sample containing the analyte for about 120 minutes.

35. The method of claim 1 wherein the sample containing the first immune complex is contacted with the second antibody bound to a solid support for about 30 minutes to one hour.

36. The method of claim 1 wherein the sample containing the first immune complex is contacted with the second antibody bound to a solid support for about 30 minutes.
(1) contacting the sample containing the analyte with:
(a) a reporter conjugate present at a concentration ranging from about 3 to 10 pM or from about 0.45 to 1.5 ng/mL, wherein the reporter conjugate comprises:
(i) a reporter antibody capable of specifically binding the analyte in the test sample with a dissociation rate constant lower than about 3.0×10⁻⁴ sec⁻¹; and
(ii) a nucleic acid marker;
thereby forming a first immune complex; and
(2) contacting the first immune complex with a capture antibody bound to a solid support, where the capture antibody is capable of specifically binding to the analyte in the first immune complex, thereby forming a two-site immune complex bound to the support; and
(3) detecting the presence and amount of the nucleic acid marker in the two-site immune complex, wherein the assay detects analyte present in the sample at concentrations of less than about 10 pg/mL.

37. The method of claim 1 wherein the detecting further comprises the use of software for the detection of nucleic acid marker amplicons to indicate the presence and amount of the analyte

38. A method for detecting a non-nucleic acid analyte present in a sample to be tested using a forward two-site immuno-PCR assay, the method comprising the steps of:
(1) contacting the sample containing the analyte with:
(a) a reporter conjugate present at a concentration ranging from about 3 to 30 pM or from about 0.45 to 4.5 ng/mL, wherein the reporter conjugate comprises:
(i) a reporter monoclonal antibody capable of specifically binding the analyte in the test sample with a dissociation rate constant lower than about 3.0×10⁻⁴ sec⁻¹; and
(ii) a nucleic acid marker;
thereby forming a first immune complex; and
(2) contacting the first immune complex with a capture monoclonal antibody bound to a solid support, where the second antibody is capable of specifically binding to the analyte in the first immune complex, thereby forming a two-site immune complex bound to the support;
(3) washing the two-site immune complex bound to the support; and
(4) detecting the presence and amount of the nucleic acid marker in the two-site immune complex, wherein the assay detects analyte present in the sample at concentrations of less than 10 pg/mL.

39. A method for detecting a non-nucleic acid analyte present in a sample to be tested using a forward two-site immuno-PCR assay, the method comprising the steps of:
(1) contacting the sample containing the analyte with:
(a) a reporter conjugate present at a concentration ranging from about 3 to 10 pM or from about 0.45 to 1.5 ng/mL, wherein the reporter conjugate comprises:
(i) a reporter monoclonal antibody capable of specifically binding the analyte in the test sample; and
(ii) a nucleic acid marker;
thereby forming a first immune complex; and
(2) contacting the first immune complex with a capture monoclonal antibody bound to a solid support, where the second antibody is capable of specifically binding to the analyte in the first immune complex, thereby forming a two-site immune complex bound to the support;
(3) washing the two-site immune complex bound to the support; and
(4) detecting the presence and amount of the nucleic acid marker in the two-site immune complex, wherein the assay detects analyte present in the sample at concentrations of less than 10 pg/mL.

40. A method for screening antibodies for use as a reporter antibody in a forward, two-site immuno-PCR assay, the method comprising
(1) obtaining the dissociation constant for two or more antibodies;
(2) performing the method of claim 1 with at least the following two antibodies used as reporter antibodies in each assay:
(a) the antibody with the lowest dissociation rate constant; and
(b) the antibody with the second lowest dissociation rate constant; and
(3) determining that the method of claim 1 performed with the antibody with the lowest dissociation constant has a higher sensitivity than the method of claim 1 performed with the antibody with the second lowest dissociation constant.

41. A method for screening antibodies for use as a reporter antibody in a forward, two-site immuno-PCR assay, the method comprising
(1) obtaining the dissociation constant for two or more antibodies;
(2) performing the forward two-site immunoassays of this invention with at least the following two antibodies used as reporter antibodies in each assay:
(a) an antibody with a k_{d} value of less than about 3×10⁻⁴ sec⁻¹; and
(b) an antibody a k_{d} value greater than about 3×10⁻⁴ sec⁻¹; and
(3) determining that the methods of the invention performed with the screened antibody with a k_{d} value of less than about 3×10⁻⁴ sec⁻¹ has a higher sensitivity than the methods of the invention performed with the screened antibody with a k_{d} value greater than 3×10⁻⁴ sec⁻¹.

42. A kit for detecting a non-nucleic acid analyte comprising:
(1) a first container comprising a reporter monoclonal capable of specifically binding to an analyte and having a dissociation rate constant lower than about 3×10⁻⁴ sec⁻¹, wherein the reporter monoclonal antibody is attached to an assay specific DNA label;
(2) a second container containing a capture monoclonal antibody for the analyte; wherein the capture monoclonal antibody is attached to a solid support.
